(19) United States
(12) Patent Application Publication
(10) Pub. No.: US 2024/0059727 A1
(43) Pub. Date: Feb. 22, 2024

(54) METHOD OF MAKING NICOTINAMIDE RIBOFURANOSIDE SALTS BY SALT METATHESIS, THE CRYSTALLINE FORM OF ITS TOSYLATE SALT AND THE CO-CRYSTALLIZED FORM OF ITS CHLORIDE:IODIDE SALT

(71) Applicant: BIOSYNTH AG, Staad (CH)

(72) Inventors: Urs Spitz, St. Gallen (CH); Aysel SOYDEMIR, Rorschach (CH); Iris ZIMMERMANN, Bregenz (AT); Günter SCHABERT, Goldach (CH)

(21) Appl. No.: 18/271,401

(22) PCT Filed: Jan. 19, 2022
(86) PCT No.: PCT/EP2022/051085

§ 371 Date: Jul. 7, 2023

(30) Foreign Application Priority Data
Jan. 19, 2021 (EP) 21152346.9
Jun. 29, 2021 (EP) 21182329.9

(51) Int. Cl. C07H 17/02

(52) U.S. Cl. CPC C07H 17/02

(57) ABSTRACT

The present invention relates to methods of making a nicotinamide ribofuranoside salt, in particular a crystalline nicotinamide ribofuranoside salt, via salt metathesis comprising subjecting nicotinamide ribofuranoside hydrogen malate or nicotinamide ribofuranoside hydrogen tartrate to salt metathesis to afford the nicotinamide ribofuranoside salt. In an alternative, acylated nicotinamide ribofuranoside hydrogen malate or acylated nicotinamide ribofuranoside hydrogen tartrate is subjected to salt metathesis followed by deacylation to afford the nicotinamide ribofuranoside salt.

### FIELD OF THE INVENTION

The present invention relates to a method of making nicotinamide-β-D-ribofuranoside salts, such as nicotinamide-β-D-ribofuranoside chloride, from nicotinamide-β-D-ribofuranoside hydrogen malate, nicotinamide-β-D-ribofuranoside hydrogen tartrate, nicotinamide-2,3,5-tri-O-acyl-β-D-ribofuranoside hydrogen malate or nicotinamide-2,3,5-tri-O-acyl-β-D-ribofuranoside hydrogen tartrate. The method of the present invention allows obtaining nicotinamide-β-D-ribofuranoside salts in a simple manner and in high yield, purity and stereoselectivity. The method also allows the preparation of co-crystallized nicotinamide-β-D-ribofuranoside (chloride, iodide).

### BACKGROUND OF THE INVENTION

Nicotinamide riboside (NR or NR⁺, nicotinamide-β-D-ribofuranoside; CAS no 1341-23-7)

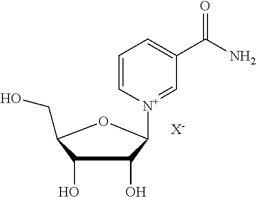

is a precursor of nicotinamide adenine dinucleotide (NAD⁺/NADH) and nicotinamide adenine dinucleotide phosphate (NADP⁺/NADPH). In addition, nicotinamide riboside is a niacin (vitamin B3) equivalent.

Nicotinamide riboside has been reported to increase NAD⁺ levels in liver and skeletal muscle and to prevent body weight gain in mice fed at high-fat diet. It also increases NAD⁺ concentration in the cerebral cortex and reduces cognitive deterioration in a transgenic mouse model of Alzheimer's disease. For these reasons, nicotinamide riboside salts have been suggested for use in nutritional supplements and pharmaceutical compositions.

The synthesis and handling of NR⁺ salts are challenging due to a relatively labile glycosidic bond compared to other nucleosides. Until now, only the bromide and the chloride salts of NR⁺ have been described in the form of crystalline salts. Whilst the crystalline bromide salt is toxic and therefore unsuitable for use as a food additive it can be and is used as starting material in chemical synthesis. The crystalline chloride salt, on the other hand, is conveniently used in food supplements and as pharmaceutically active ingredient.

Current production methods of NR⁺ salts often produce low yields, which is a huge challenge for large-scale production. Furthermore, these methods often suffer from poor stereoselectivity, resulting in the formation of a mixture of the α and β anomers of NR⁺. Since only the β form is biologically active this is highly undesirable for certain uses. Further drawbacks of known production methods of NR⁺ salts include the use of expensive reagents, thereby rendering the method uneconomical for commercial production. In addition, the frequently used ion exchangers have a low exchange capacity and are therefore unfavorable for production of NR⁺ salts in large amounts. Also, the use of large quantities of solvents promotes the undesirable hydrolysis of NR⁺ salts. Thus, the methods known in the prior art for preparing NR salts such as NR chloride have drawbacks, especially when applied to large-scale commercial production.

WO 2017/218580 discloses synthetic methods for the preparation of nicotinamide riboside salts that involve replacing a pharmaceutically acceptable counter-ion of a nicotinamide riboside salts by another pharmaceutically acceptable counter-ion, e.g., the chloride anion, through ion exchange chromatography or salt exchange reaction and precipitation to give the desired salt of NR and pharmaceutically acceptable counter-ion, e.g., the NR chloride salt. WO 2015/186068 discloses the reaction of nicotinamide-β-D-ribofuranoside triflate with sodium methylate in an ion exchange reaction to afford crystalline nicotinamide-β-D-riboside chloride. Furthermore, CN 108774278 discloses the deacetylation of nicotinamide triacetylribofuranoside triflate using a base, followed by the treatment of the deacetylated product with an acid to yield the corresponding salt product.

### OBJECT OF THE INVENTION

In view of the above, there is a need in the art for a method of making nicotinamide ribofuranoside salts, especially pharmaceutically acceptable salts of nicotinamide ribofuranoside such as the chloride salt, in a simple and cost-efficient manner at high yield, purity and stereoselectivity on a commercial scale.

### SUMMARY OF THE INVENTION

Surprisingly, it has been found that this object can be achieved by using nicotinamide-β-D-ribofuranoside hydrogen malate or nicotinamide-β-D-ribofuranoside hydrogen tartrate as starting materials, preferably in crystalline form, and subjecting these hydrogen malate or hydrogen tartrate salts to salt metathesis comprising counter-ion exchange with a given anion, e.g. chloride anion, to afford the desired nicotinamide-β-D-ribofuranoside salt, e.g. the nicotinamide-β-D-ribofuranoside chloride salt.

This method is simple and cost-efficient and obtains the desired nicotinamide-β-D-ribofuranoside salt, e.g., the nicotinamide-p-D-ribofuranoside chloride salt, in high yield, purity and stereoselectivity. Furthermore, the desired nicotinamide-β-D-ribofuranoside salt can be advantageously obtained in crystalline form, which crystalline salts are particularly useful in nutritional and pharmaceutical applications.

In an alternative method, nicotinamide-2,3,5-tri-O-acyl-β-D-ribofuranoside hydrogen malate or nicotinamide-2,3,5-tri-O-acyl-β-D-ribofuranoside hydrogen tartrate is subjected to salt metathesis and the acyl groups are cleaved to afford the desired nicotinamide-β-D-ribofuranoside salt.

According to a first aspect, the invention relates to a method of making a nicotinamide-β-D-ribofuranoside salt, comprising step (A):
(A) subjecting nicotinamide-β-D-ribofuranoside hydrogen malate or nicotinamide-β-D-ribofuranoside hydrogen tartrate to salt metathesis comprising counter-ion exchange to afford the nicotinamide-β-D-ribofuranoside salt.

According to a second aspect, the invention relates to a method of making a nicotinamide-β-D-ribofuranoside salt, comprising steps (A) and (B):
(A) subjecting nicotinamide-2,3,5-tri-O-acyl-β-D-ribofuranoside hydrogen malate or nicotinamide-2,3,5-tri-O-acyl-β-D-ribofuranoside hydrogen tartrate to salt metathesis comprising counter-ion exchange to afford a nicotinamide-2,3,5-tri-O-acyl-β-D-ribofuranoside salt;
(B) deacylating the nicotinamide-2,3,5-tri-O-acyl-β-D-ribofuranoside salt to afford the nicotinamide-p-D-ribofuranoside salt;
wherein steps (A) and (B) are carried out simultaneously or step (B) is carried out subsequently to step (A).

Preferred embodiments are defined in the appended claims.

### BRIEF DESCRIPTION OF THE FIGURES

The present invention is further described by the appended figures, in which:

FIG. 1 shows a powder X-ray pattern of crystalline nicotinamide-β-D-ribofuranoside D-hydrogen malate;

FIG. 2 shows a powder X-ray pattern of crystalline nicotinamide-β-D-ribofuranoside L-hydrogen malate;

FIG. 3 shows a powder X-ray pattern of crystalline nicotinamide-β-D-ribofuranoside DL-hydrogen malate;

FIG. 4 shows a powder X-ray pattern of crystalline nicotinamide-β-D-ribofuranoside D-hydrogen tartrate monohydrate;

FIG. 5 shows a powder X-ray pattern of crystalline nicotinamide-β-D-ribofuranoside L-hydrogen tartrate;

FIG. 6 shows a powder X-ray pattern of crystalline nicotinamide-β-D-ribofuranoside DL-hydrogen tartrate;

FIG. 7 shows a powder X-ray pattern of crystalline nicotinamide-2,3,5-O-triacetyl-β-D-ribofuranoside L-hydrogen tartrate;

FIG. 8 shows a powder X-ray pattern of crystalline nicotinamide-2,3,5-O-triacetyl-β-D-ribofuranoside D-hydrogen tartrate;

FIG. 9 shows a powder X-ray pattern of crystalline anhydrous nicotinamide-β-D-ribofuranoside D-hydrogen tartrate;

FIG. 10 shows a powder X-ray pattern of crystalline nicotinamide-β-D-ribofuranoside tosylate; and

FIG. 11 shows a powder X-ray pattern of co-crystallized nicotinamide-β-D-ribofuranoside (chloride, iodide), wherein chloride and iodide are present in a molar ratio of 2:1.

{x-axis: Position [°2Theta] (Copper(Cu); y-axis: Counts), respectively}.

### DETAILED DESCRIPTION OF THE INVENTION

The various aspects of the present invention will now be described in more detail with reference to the accompanying figures.

### First and Second Aspect: Methods According to the Invention

According to a first aspect, the invention relates to a method of preparing a nicotinamide-β-D-ribofuranoside salt by replacing the anion X⁻=hydrogen malate or hydrogen tartrate in a compound of formula

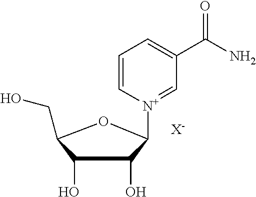

through an anion Y⁻ via salt metathesis comprising counter-ion exchange to afford NR⁺Y⁻ Preferably, the anion Y⁻ is chloride. However, the method is not restricted thereto.

Accordingly, in a first aspect, the invention relates to a method of making a nicotinamide-β-D-ribofuranoside salt, comprising step (A):
(A) subjecting nicotinamide-β-D-ribofuranoside hydrogen malate or nicotinamide-β-D-ribofuranoside hydrogen tartrate to salt metathesis comprising counter-ion exchange to afford the nicotinamide-β-D-ribofuranoside salt.

According to a second aspect, nicotinamide-2,3,5-tri-O-acyl-β-D-ribofuranoside hydrogen malate or nicotinamide-2,3,5-tri-O-acyl-β-D-ribofuranoside hydrogen tartrate of formula

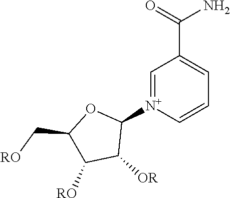

X⁻=hydrogen malate or hydrogen tartrate is subjected to salt metathesis to exchange X⁻ through an anion Depending on the reaction conditions, in one embodiment, steps (A) and (B) may proceed simultaneously, i.e. the acyl groups may be cleaved simultaneously to the formation of the desired nicotinamide-β-D-ribofuranoside salt NR⁺Y⁻. In another embodiment, step (B) is carried out subsequently to step (A). Preferably, the anion Y⁻ is chloride. However, the method is not restricted thereto.

Accordingly, in the second aspect, the invention relates to a method of making a nicotinamide-β-D-ribofuranoside salt, comprising steps (A) and (B):
(A) subjecting nicotinamide-2,3,5-tri-O-acyl-β-D-ribofuranoside hydrogen malate or nicotinamide-2,3,5-tri-O-acyl-β-D-ribofuranoside hydrogen tartrate to salt metathesis comprising counter-ion exchange to afford a nicotinamide-2,3,5-tri-O-acyl-β-D-ribofuranoside salt; and
(B) deacylating the nicotinamide-2,3,5-tri-O-acyl-β-D-ribofuranoside salt to afford the nicotinamide-p-D-ribofuranoside salt;
wherein steps (A) and (B) are carried out simultaneously or step (B) is carried out subsequently to step (A).

The term “acyl” as used in connection with nicotinamide-2,3,5-tri-O-acyl-β-D-ribofuranoside salts means an acyl group that is independently selected from alkyl carbonyl, aryl carbonyl and heteroaryl carbonyl, preferably from C₁₋₁₀ alkyl carbonyl and benzoyl, and is more preferably acetyl, and wherein said acyl groups are optionally independently substituted with one or more substituents selected from: C₁₋₆ alkyl, C₁₋₆ alkoxy, C₁₋₆ thioalkyl, halogen, nitro, cyano, NH(C₁₋₆ alkyl), N(C₁₋₆ alkyl)₂, and SO₂N(C₁₋₆ alkyl)₂.

Preferably, the hydrogen malate is D-, L- or DL-hydrogen malate. Further, the hydrogen tartrate is preferably D-, L- or DL-hydrogen tartrate. Advantageously, D-, L- or DL-hydrogen malate or D-, L- or DL-hydrogen tartrate may be provided in high purity and high stereoselectivity in terms of β anomers.

Moreover, the salts used in step (A) of the method according to the first aspect of the present invention, i.e., nicotinamide-β-D-ribofuranoside hydrogen malate or nicotinamide-p-D-ribofuranoside hydrogen tartrate, or both, may be crystalline salts. Likewise, the salts used in step (A) of the method according to the second aspect of the present invention, i.e. nicotinamide-2,3,5-tri-O-acyl-β-D-ribofuranoside hydrogen malate or nicotinamide-2,3,5-tri-O-acyl-β-D-ribofuranoside hydrogen tartrate, or both, may be crystalline salts. Within the present invention, the use of crystalline salts is preferred since it allows for the manufacture of crystalline NR⁺ salts of particularly high purity and stereoselectivity in terms of the R anomer.

The term “salt metathesis”, as used herein, is synonymously used with terms such as “double replacement reaction”, “double displacement reaction” or “double decomposition reaction”. Salt metathesis for exchanging counter-ions between two different salts is a known technique. It should be understood that the term “salt metathesis” does not mean that the anion of the p-nicotinamide riboside is exchanged by another anion by means of ion exchange using an ion exchanger. Thus, the methods according to the invention defined in step (A) excludes an anion exchange by means of an ion exchanger. However, the method does not exclude that in any reaction step prior to step (A) or subsequently to step (A) an ion exchanger is used.

Step (A) defines a reaction, wherein a first salt, i.e. a nicotinamide-β-D-ribofuranoside salt NR⁺X⁻ or a nicotinamide-2,3,5-tri-O-acyl-β-D-ribofuranoside salt AcONR⁺X⁻ is subjected to a salt metathesis using a suitable compound to provide an anion Y⁻, which compound is Cat⁺Y⁻ comprising a cation Cat⁺ and said anion Y⁻, to afford a nicotinamide-β-D-ribofuranoside salt NR⁺Y⁻ or a nicotinamide-2,3,5-tri-O-acyl-β-D-ribofuranoside salt AcONR⁺Y⁻ and Cat⁺X⁻ via counter-ion exchange, i.e. exchange of X⁻ in NR⁺X⁻ or AcONR⁺X⁻ by Y⁻. These reactions are summarized by the following equations:

NR⁺X⁻+Cat⁺Y⁻→NR⁺Y⁻+Cat⁺X⁻

AcONR⁺X⁻+Cat⁺Y⁻→AcONR⁺Y⁻+Cat⁺X⁻,

wherein said compound Cat⁺Y⁻ may be a suitable salt or a suitable acid.

The driving force of a salt metathesis reaction such as the reactions described above may be the formation of more stable salts as well as the removal of a product from the chemical equilibrium of the reaction, e.g., by precipitation of one of the formed NR⁺Y⁻ and Cat⁺X⁻ or one of the formed AcONR⁺Y⁻ and Cat⁺X⁻. Thus, in order to drive the reaction to the products, the educts should be selected in view of solubility in one another or in a solvent, respectively in view of favorable energies.

In a preferred embodiment, nicotinamide-β-D-ribofuranoside hydrogen malate or hydrogen tartrate or nicotinamide-2,3,5-tri-O-acyl-β-D-ribofuranoside hydrogen malate or hydrogen tartrate are reacted with an acid, e.g., an organic or an inorganic acid, to effect the anion exchange. Accordingly, in this embodiment, Cat⁺ is H⁺. The acid is preferably a strong acid. The term “strong acid”, as used herein, means that the acid is a stronger acid than malic acid or tartaric acid. Preferably, said strong acid has a pKₐ below 2, further preferred below 1, or still more preferred below 0.

Preferably, the acid Cat⁺Y⁻=H⁺Y⁻ is used in a molar excess compared to the starting material nicotinamide-β-D-ribofuranoside hydrogen malate or hydrogen tartrate or nicotinamide-2,3,5-tri-O-acyl-β-D-ribofuranoside hydrogen malate or hydrogen tartrate. Preferably, more than 1.1 molar equivalents of acid H⁺Y⁻ are used, further preferred at least 1.2 or at least 1.3 or at least 1.4 or at least 1.5 equivalents.

If Cat⁺ is H⁺, i.e. an acid H⁺Y⁻ is used for counter-ion exchange, steps (A) and (B) in the method according to the second aspect typically proceed simultaneously, i.e. cleavage of the acyl groups in the starting material nicotinamide-2,3,5-tri-O-acyl-β-D-ribofuranoside hydrogen malate or hydrogen tartrate and/or formed nicotinamide-2,3,5-tri-O-acyl-β-D-ribofuranoside salt and formation of the desired nicotinamide-β-D-ribofuranoside salt may proceed simultaneously.

If Cat⁺ is not H⁺, i.e. not an acid H⁺Y⁻ but a salt Cat⁺Y⁻ is employed in the method according to the second aspect of the present invention, steps (A) and (B) typically proceed in successive steps, i.e. cleavage of the acyl groups from the nicotinamide-2,3,5-tri-O-acyl-β-D-ribofuranoside salt (step (B)) is conducted after formation of nicotinamide-2,3,5-tri-O-acyl-β-D-ribofuranoside salt by metathesis (step (A)). Cleavage of the acyl groups may be performed in accordance with methods known in the art, e.g., by subjecting the nicotinamide-2,3,5-tri-O-acyl-β-D-ribofuranoside salt obtained in step (A) to an acid such as hydrogen bromide, hydrogen chloride, hydrogen iodide or sulfuric acid, or to a base such as ammonia.

According to the second aspect of the present invention, the nicotinamide-2,3,5-tri-O-acyl-β-D-ribofuranoside salt obtained in step (A) may be isolated and purified before it is deacylated in step (B). Alternatively, the nicotinamide-2,3,5-tri-O-acyl-β-D-ribofuranoside salt of step (A) may not be purified prior to deacylation in step (B).

In accordance with the present invention, the counter-ion (Y⁻) of the salt obtained in step (A) via counter-ion exchange may be selected from the group consisting of:
inorganic ions;
carboxylates, optionally substituted with one or more substituents independently selected from the group consisting of carboxyl, hydroxyl, thio, keto, amino, mono C₁₋₆ alkyl, hydroxy C₁₋₆ alkylene and di(C₁₋₆ alkyl) amino;
C₁₋₁₂ alkyl sulfonates; or
arylsulfonates, wherein the aryl moiety is optionally substituted with one or more substituents independently selected from the group consisting of carboxyl, hydroxyl, amino, mono-C₁₋₆ alkyl and di(C₁₋₆ alkyl)amino, halogen, and C₁₋₆ alkyl; and
wherein Y⁻ is not hydrogen malate or hydrogen tartrate.

The inorganic ion may be selected from the group consisting of bromide, chloride, iodide, hydrogen sulfate, sulfate, dihydrogen phosphate, monohydrogen phosphate, phosphate;
the carboxylate may be selected from the group consisting of formate, acetate, oxalate, malonate, succinate, fumarate, maleate, citrate, ascorbate, β-ketoglutarate, glucuronate, benzoate and salicylate;
the C₁₋₁₂ alkylsulfonate may be selected from the group consisting of mesylate and camsylate; and
the arylsulfonate may be selected from the group consisting of besylate and tosylate.

Preferably, the counter-ion Y⁻ is selected from chloride and bromide, preferably from hydrogen chloride or hydrogen bromide used for effecting counter-ion exchange by salt metathesis. More preferably, the counter-ion is chloride, in particular from hydrogen chloride used for effecting counter-ion exchange by salt metathesis.

The salt metathesis may be performed without a solvent, i.e. via salt metathesis of a solid nicotinamide-β-D-ribofuranoside salt or solid nicotinamide-2,3,5-tri-O-acyl-β-D-ribofuranoside salt with, e.g., a liquid salt or a liquid acid. However, the salt metathesis in step (A) is preferably performed in the presence of a solvent.

In the following, four preferred embodiments for performing the above-defined salt metathesis reaction are described.

Embodiment I: The solvent is selected such that NR⁺X⁻ (or AcONR⁺X⁻) and Cat⁺Y⁻ are both soluble in said solvent, however NR⁺Y⁻ (or AcONR⁺Y⁻) obtained in step (A) is not soluble in said solvent and precipitates, whereas Cat⁺X⁻ is soluble. In this case, NR⁺Y⁻ (or AcONR⁺Y⁻) may be isolated by filtration.

Embodiment II: The solvent is selected such that NR⁺X⁻ (or AcONR⁺X⁻) and Cat⁺Y⁻ are both soluble in said solvent, however NR⁺Y⁻ (or AcONR⁺Y⁻) obtained in step (A) is soluble in said solvent, whereas Cat⁺X⁻ is not soluble and precipitates. In this case, NR⁺Y⁻ (or AcONR⁺Y⁻) may be isolated from the supernatant according to known techniques.

Embodiment III: The solvent is selected such that NR⁺X⁻ and NR⁺Y⁻ of step (A) (or AcONR⁺X⁻ and AcONR⁺Y⁻) are both not soluble in said solvent, whereas both Cat⁺X⁻ and Cat⁺Y⁻ are soluble. In this case, NR⁺Y⁻ (or AcONR⁺Y⁻) may be isolated by, e.g., filtration. Embodiment III gives particularly good results if Cat⁺Y⁻ is an acid as defined above, and, preferably, the solvent is an alcohol.

Embodiment IV: The solvent is selected such that NR⁺X⁻ and NR⁺Y⁻ (or AcONR⁺X⁻ and AcONR⁺Y⁻) of step (A) are soluble in said solvent, whereas Cat⁺Y⁻ and Cat⁺X⁻ are not soluble. NR⁺Y⁻ (or AcONR⁺Y⁻) may e.g. then be isolated from the supernatant according to known techniques.

Preferably, the solvent used in step (A) of the methods according to the present invention is an alcohol selected from the group consisting of methanol, ethanol, propanol (e.g., n-propanol, iso-propanol), or butanol (e.g., n-butanol, iso-butanol, sec-butanol, tert-butanol), or a mixture of two or more thereof, optionally wherein the alcohol or the mixture of alcohol comprises water.

Accordingly, by appropriate choice of the solvent used in the salt metathesis reaction defined in step (A) the desired salt can be obtained and isolated.

Preferably, in step (A) a suspension of nicotinamide-β-D-ribofuranoside hydrogen malate or nicotinamide-β-D-ribofuranoside hydrogen tartrate or nicotinamide-2,3,5-tri-O-acyl-β-D-ribofuranoside hydrogen malate or nicotinamide-2,3,5-tri-O-acyl-β-D-ribofuranoside hydrogen tartrate in one or more of the alcohols defined above, optionally comprising water, and a suitable acid are combined with one another to carry out step (A), i.e. the nicotinamide-β-D-ribofuranoside salt or the nicotinamide-2,3,5-tri-O-acyl-β-D-ribofuranoside salt (which may already be deacylated to give the nicotinamide-β-D-ribofuranoside salt) is formed by counter-ion exchange and typically precipitates so that it can be isolated, for example, by filtration.

The isolated nicotinamide-p-β-ribofuranoside salt or the nicotinamide-2,3,5-tri-O-acyl-β-D-ribofuranoside salt are generally obtained in crystalline form, high purity and high stereoselectivity in terms of β anomer. Thus, crystalline compounds are obtained directly in the salt metathesis reaction without the need of using an ion-exchanger or using complex purification and/or crystallization methods. This is a major advantage of the method in accordance with the present invention compared to, e.g., a counter-ion exchange via ion-exchanger where the compounds typically are obtained in an amorphous form and need to be crystallized in subsequent steps to obtain the desired purity and stereoisomer. Notwithstanding the above, it is contemplated that the products obtained in accordance with the methods of the present invention may be further purified by a crystallization step, if desired.

Preferably, the salt metathesis reaction according to step (A) is performed at ambient temperature, i.e., in the range of from 5 to 60° C., preferably at a temperature of 10 to 40° C.

The manufacture of NR⁺ D-, L- or DL-hydrogen malate or NR⁺ D-, L- or DL-hydrogen tartrate, or AcONR⁺ D-, L- or DL-hydrogen malate or AcONR⁺ D-, L- or DL-hydrogen tartrate, is described in more detail hereinunder.

### Preparation of Nicotinamide-β-D-Ribofuranoside Hydrogen Malate or Hydrogen Tartrate or Nicotinamide-2,3,5-Tri-O-Acyl-β-D-Ribofuranoside Hydrogen Malate or Hydrogen Tartrate

The nicotinamide-β-D-ribofuranoside hydrogen malate or hydrogen tartrate salt or nicotinamide-2,3,5-tri-O-acyl-β-D-ribofuranoside hydrogen malate or hydrogen tartrate salt used as starting material in step (A) is made by salt metathesis comprising counter-ion exchange of a nicotinamide-p-D-ribofuranoside bromide, chloride, iodide, triflate, nonaflate, fluorosulfonate or perchlorate or nicotinamide-2,3,5-tri-O-acyl-β-D-ribofuranoside bromide, chloride, iodide, triflate, nonaflate, fluorosulfonate or perchlorate with hydrogen malate or hydrogen tartrate.

Nicotinamide-β-D-ribofuranoside bromide is a well-known compound (CAS no 78687-39-5). E.g., Lee et al. disclose a chemical synthesis method thereof (Chem. Commun., 1999, 729-730). Said reference also discloses the preparation of nicotinamide-2,3,5-tri-O-acyl-β-D-ribofuranoside bromide as a precursor of nicotinamide-β-D-ribofuranoside bromide.

Nicotinamide-β-D-ribofuranoside triflate is also a well-known compound (CAS no 445489-49-6). Nicotinamide-β-D-ribofuranoside triflate and nicotinamide-2,3,5-tri-O-acyl-β-D-ribofuranoside triflate may be prepared, e.g., by reacting nicotinamide with a tetra-O-acyl-β-D-ribofuranose in acetonitrile in the presence of trimethylsilyl trifluoromethanesulfonate (TMSOTf) to afford a nicotinamide-2,3,5-tri-O-acyl-β-D-ribofuranoside triflate. The acyl groups may then be cleaved according to known methods to afford the nicotinamide-β-D-ribofuranoside triflate (see e.g., Makarova et al.: “Syntheses and chemical properties of β-nicotinamide riboside and its analogues and derivatives”, Beilstein J Org Chem 2019, 15: 401-430; Tanimori et al., “An Efficient Chemical Synthesis of Nicotinamide Riboside (NAR) and Analogues”, Bioorganic & Medicinal Chemistry Letters 12 (2002) 1135-1137).

Nicotinamide-β-D-ribofuranoside nonaflate and nicotinamide-2,3,5-tri-O-acyl-β-D-ribofuranoside nonaflate, respectively nicotinamide-β-D-ribofuranoside perchlorate and nicotinamide-2,3,5-tri-O-acyl-β-D-ribofuranoside perchlorate, may be prepared by reacting nicotinamide with a tetra-O-acyl-β-D-ribofuranose in a solvent such as acetonitrile in the presence of trimethylsilyl nonafluorobutanesulfonate (CAS no 68734-62-3), respectively trimethylsilyl perchlorate (CAS no 18204-79-0) to afford a nicotinamide-2,3,5-tri-O-acyl-β-D-ribofuranoside nonaflate, respectively perchlorate. The acyl groups may then be cleaved according to known methods to afford the nicotinamide-β-D-ribofuranoside nonaflate, respectively perchlorate.

Nicotinamide-β-D-ribofuranoside chloride and nicotinamide-2,3,5-tri-O-acyl-β-D-ribofuranoside chloride, nicotinamide-β-D-ribofuranoside iodide and nicotinamide-2,3,5-tri-O-acyl-β-D-ribofuranoside iodide, respectively nicotinamide-p-D-ribofuranoside fluorosulfonate and nicotinamide-2,3,5-tri-O-acyl-β-D-ribofuranoside fluorosulfonate, may be prepared by reacting nicotinamide with a tetra-O-acyl-β-D-ribofuranose in a solvent such as acetonitrile in the presence of trimethylsilyl choride (CAS no 75-77-4), trimethylsilyl iodide (CAS no. 16029-98-4), respectively trimethylsilyl fluorosulfonate (CAS no 3167-56-4) to afford a nicotinamide-2,3,5-tri-O-acyl-β-D-ribofuranoside chloride, iodide, and fluorosulfonate, respectively. The acyl groups may then be cleaved according to known methods to afford the nicotinamide-β-D-ribofuranoside chloride, iodide, and fluorosulfonate, respectively.

Nicotinamide-2,3,5-tri-O-acyl-β-D-ribofuranoside bromides, nicotinamide-2,3,5-tri-O-acyl-β-D-ribofuranoside chlorides, nicotinamide-2,3,5-tri-O-acyl-β-D-ribofuranoside iodides, nicotinamide-2,3,5-tri-O-acyl-β-D-ribofuranoside trifluoromethanesulfonates, nicotinamide-2,3,5-tri-O-acyl-β-D-ribofuranoside nonafluorobutanesulfonates, nicotinamide-2,3,5-tri-O-acyl-β-D-ribofuranoside fluorosulfonates or nicotinamide-2,3,5-tri-O-acyl-β-D-ribofuranoside perchlorates for making the respective hydrogen malates and hydrogen tartrates used in step (A) of the method according to the second aspect are either known or, as described above, can be prepared according to known methods.

In order to obtain the hydrogen malate or hydrogen tartrate used in step (A) of the methods according to the present invention, nicotinamide-β-D-ribofuranoside bromide, chloride, iodide, triflate, nonaflate, fluorosulfonate or perchlorate or nicotinamide-2,3,5-tri-O-acyl-β-D-ribofuranoside bromide, chloride, iodide, triflate, nonaflate, fluorosulfonate or perchlorate may be reacted with a salt of the hydrogen malate or hydrogen tartrate in a salt metathesis reaction.

Ammonium salts thereof such as trialkyl ammonium salts are particularly useful, e.g., triethyl ammonium salts or tributyl ammonium salts.

The term “hydrogen malate” as used herein means the monocarboxylate. Preferably, the hydrogen malate is the D-, L- or DL-stereoisomer. As used herein, the term “hydrogen tartrate” means the monocarboxylate. Preferably, the hydrogen tartrate ion is the D-, L- or DL-stereoisomer.

The preparation of NR or AcONR D-, L- or DL-stereoisomers of hydrogen malate or hydrogen tartrate is advantageous since the method according to the present invention beneficially allows for the provision of these compounds in a high yield and in crystalline form, which is particularly advantageous in view of the handling and further processing of the salt.

Typically, crystalline compounds are already obtained directly in the salt metathesis reaction without the need of using an ion-exchanger or using complex purification and/or crystallization methods. For example, starting from nicotinamide-β-D-ribofuranoside bromide (NR⁺Br⁻) the following crystalline nicotinamide-β-D-ribofuranoside hydrogen malates and nicotinamide-β-D-ribofuranoside hydrogen tartrates may be obtained via salt metathesis in excellent purity:

| | Purity | Residual Br |
|---|---|---|
| Salt | (determined | (determined by ion exchange |
| (NR⁺X⁻) | by NMR) | chromatography IC) |
| D-hydrogen tartrate | >97% | 0.3% |
| L-hydrogen tartrate | >97% | 0.2% |
| DL-hydrogen tartrate | >97% | 0.1% |
| L-hydrogen malate | >97% | 0.1% |
| D-hydrogen malate | >97% | 0.9% |
| DL-hydrogen malate | >96% | 2.3% |

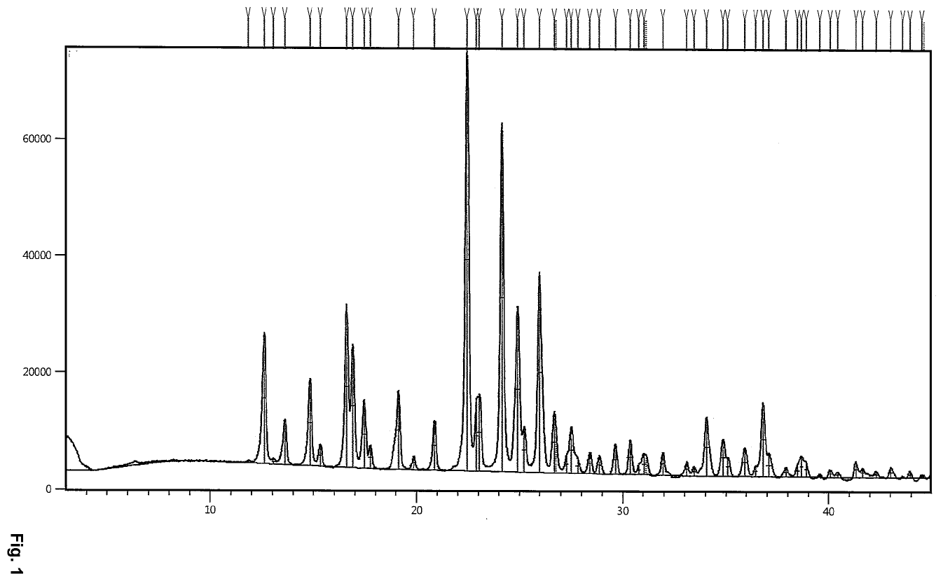

In a preferred embodiment, the crystalline nicotinamide-β-D-ribofuranoside hydrogen malate used in step (A) of the method according to the first aspect is nicotinamide-β-D-ribofuranoside D-hydrogen malate. This compound may be characterized by a powder X-ray diffraction pattern having peaks substantially as provided in Table 1, below, ±0.2 degrees two theta, or as provided in FIG. 1:

TABLE 1

| Pos. | Height | FWHM Left | d-spacing | Rel. Int. |
|---|---|---|---|---|
| [°2Th.] | [cts] | [°2Th.] | [Å] | [%] |
| 11.8481 | 445.20 | 0.1535 | 7.46957 | 0.61 |
| 12.6145 | 22336.51 | 0.1663 | 7.01743 | 30.71 |
| 13.0454 | 990.49 | 0.1023 | 6.78661 | 1.36 |
| 13.6055 | 7710.12 | 0.1407 | 6.50845 | 10.60 |
| 14.8288 | 14677.80 | 0.1407 | 5.97418 | 20.18 |
| 15.3150 | 3657.25 | 0.1279 | 5.78560 | 5.03 |
| 16.6048 | 27830.00 | 0.1535 | 5.33898 | 38.27 |
| 16.8966 | 21052.43 | 0.1407 | 5.24745 | 28.95 |
| 17.4485 | 11645.88 | 0.1535 | 5.08269 | 16.01 |
| 17.7534 | 3843.61 | 0.1407 | 4.99606 | 5.29 |
| 19.1083 | 13443.46 | 0.1535 | 4.64476 | 18.49 |
| 19.8613 | 2277.97 | 0.1023 | 4.47034 | 3.13 |
| 20.8582 | 8387.39 | 0.1535 | 4.25887 | 11.53 |
| 22.4545 | 72723.85 | 0.1663 | 3.95960 | 100.00 |
| 22.9093 | 12085.33 | 0.1023 | 3.88200 | 16.62 |
| 23.0396 | 13135.67 | 0.1279 | 3.86035 | 18.06 |
| 24.1487 | 59629.80 | 0.1663 | 3.68550 | 81.99 |
| 24.9146 | 28316.42 | 0.1791 | 3.57392 | 38.94 |
| 25.2144 | 7756.64 | 0.1151 | 3.53210 | 10.67 |
| 25.9681 | 34260.16 | 0.1791 | 3.43127 | 47.11 |
| 26.6864 | 10373.63 | 0.1872 | 3.33776 | 14.26 |
| 26.7773 | 7203.21 | 0.0624 | 3.33490 | 9.90 |
| 27.2747 | 3052.68 | 0.0936 | 3.26708 | 4.20 |
| 27.5074 | 7926.64 | 0.1716 | 3.23997 | 10.90 |
| 27.8283 | 2567.70 | 0.1560 | 3.20334 | 3.53 |
| 28.4116 | 3658.38 | 0.1872 | 3.13888 | 5.03 |
| 28.8702 | 3091.52 | 0.2184 | 3.09006 | 4.25 |
| 29.6423 | 5092.55 | 0.1872 | 3.01130 | 7.00 |
| 30.3593 | 5978.38 | 0.2340 | 2.94180 | 8.22 |
| 30.7689 | 1776.07 | 0.1248 | 2.90356 | 2.44 |
| 31.0365 | 3671.22 | 0.1560 | 2.87914 | 5.05 |
| 31.1463 | 3208.07 | 0.1248 | 2.87636 | 4.41 |
| 31.9512 | 3909.20 | 0.1560 | 2.79877 | 5.38 |
| 33.1011 | 2415.68 | 0.1872 | 2.70412 | 3.32 |
| 33.4482 | 1483.40 | 0.1872 | 2.67685 | 2.04 |
| 34.0593 | 9996.43 | 0.2028 | 2.63021 | 13.75 |
| 34.8632 | 6370.44 | 0.2028 | 2.57137 | 8.76 |
| 35.0955 | 3176.40 | 0.1092 | 2.55488 | 4.37 |
| 35.9193 | 4930.68 | 0.2028 | 2.49815 | 6.78 |
| 36.4557 | 1816.82 | 0.1092 | 2.46262 | 2.50 |
| 36.8016 | 12666.84 | 0.2028 | 2.44026 | 17.42 |
| 37.0816 | 3874.77 | 0.2184 | 2.42247 | 5.33 |
| 37.9077 | 1556.53 | 0.2184 | 2.37157 | 2.14 |
| 38.4661 | 2055.02 | 0.1560 | 2.33841 | 2.83 |
| 38.6659 | 3561.10 | 0.1248 | 2.32679 | 4.90 |
| 38.8999 | 2595.21 | 0.1248 | 2.31332 | 3.57 |
| 39.5529 | 483.59 | 0.1872 | 2.27663 | 0.66 |
| 40.0729 | 1196.25 | 0.1248 | 2.24827 | 1.64 |
| 40.4331 | 913.59 | 0.2028 | 2.22907 | 1.26 |
| 41.3218 | 2816.77 | 0.1872 | 2.18316 | 3.87 |

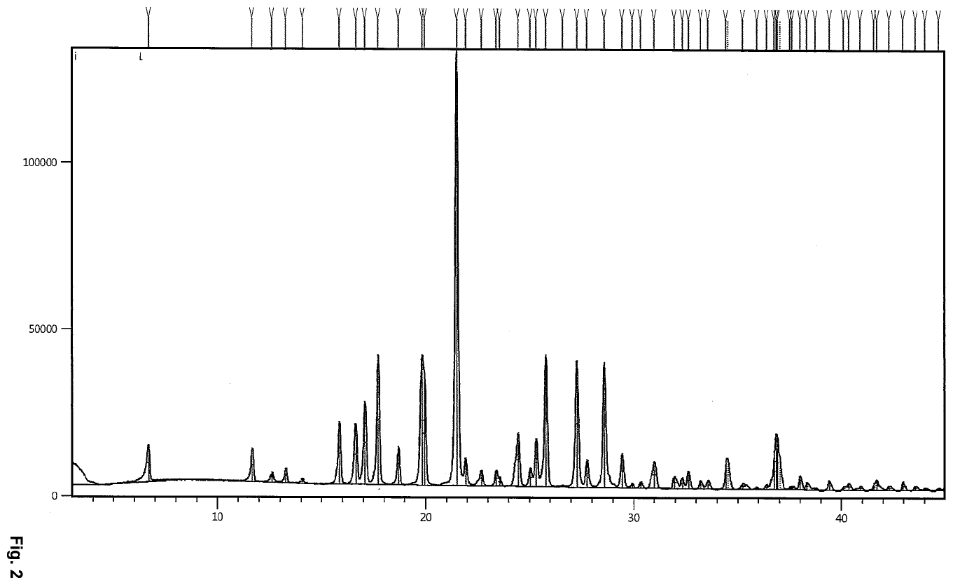

In a further preferred embodiment, the crystalline nicotinamide-β-D-ribofuranoside hydrogen malate used in step (A) of the method according to the first aspect is nicotinamide-β-D-ribofuranoside L-hydrogen malate. This compound may be characterized by a powder X-ray diffraction pattern having peaks substantially as provided in Table 2, below, ±0.2 degrees two theta, or as provided in FIG. 2:

TABLE 2

| Pos. | Height | FWHM Left | d-spacing | Rel. Int. |
|---|---|---|---|---|
| [°2Th.] | [cts] | [°2Th.] | [Å] | [%] |
| 6.6733 | 11000.76 | 0.1279 | 13.24569 | 8.35 |
| 11.6730 | 10108.79 | 0.1023 | 7.58124 | 7.67 |
| 12.6182 | 2979.47 | 0.1023 | 7.01537 | 2.26 |
| 13.2792 | 4005.91 | 0.1023 | 6.66763 | 3.04 |
| 14.0796 | 1500.07 | 0.0895 | 6.29033 | 1.14 |
| 15.8520 | 18627.05 | 0.1151 | 5.59079 | 14.14 |
| 16.6325 | 17905.54 | 0.1279 | 5.33014 | 13.59 |
| 17.0744 | 24889.77 | 0.1407 | 5.19318 | 18.90 |
| 17.7100 | 39009.26 | 0.1279 | 5.00821 | 29.62 |
| 18.6845 | 11202.45 | 0.1151 | 4.74915 | 8.50 |
| 19.8229 | 39113.57 | 0.1023 | 4.47890 | 29.70 |
| 19.9594 | 30901.54 | 0.0895 | 4.44858 | 23.46 |
| 21.4914 | 131716.20 | 0.1535 | 4.13481 | 100.00 |
| 21.9147 | 8196.29 | 0.1279 | 4.05590 | 6.22 |
| 22.6727 | 4614.03 | 0.1151 | 3.92199 | 3.50 |
| 23.3985 | 4580.59 | 0.1023 | 3.80195 | 3.48 |
| 23.5603 | 2609.14 | 0.0768 | 3.77619 | 1.98 |
| 24.4454 | 15991.37 | 0.1407 | 3.64145 | 12.14 |
| 25.0307 | 5436.84 | 0.1151 | 3.55761 | 4.13 |
| 25.3173 | 14403.35 | 0.1407 | 3.51797 | 10.94 |
| 25.7774 | 39496.78 | 0.1279 | 3.45622 | 29.99 |
| 26.5990 | 530.11 | 0.0768 | 3.35130 | 0.40 |
| 27.2687 | 37878.08 | 0.1535 | 3.27050 | 28.76 |
| 27.7679 | 8088.77 | 0.1407 | 3.21283 | 6.14 |
| 28.5924 | 37492.90 | 0.1407 | 3.12203 | 28.46 |
| 29.4458 | 10235.99 | 0.1535 | 3.03346 | 7.77 |
| 29.9251 | 1263.01 | 0.1023 | 2.98595 | 0.96 |
| 30.3346 | 1771.52 | 0.1279 | 2.94658 | 1.34 |
| 30.9962 | 7832.28 | 0.1407 | 2.88517 | 5.95 |
| 31.9611 | 3386.65 | 0.1151 | 2.80024 | 2.57 |
| 32.3205 | 3078.07 | 0.1151 | 2.76992 | 2.34 |
| 32.6276 | 5095.47 | 0.1407 | 2.74454 | 3.87 |
| 33.2109 | 2336.00 | 0.1151 | 2.69767 | 1.77 |
| 33.5827 | 2533.18 | 0.1791 | 2.66864 | 1.92 |
| 34.4375 | 8372.54 | 0.1092 | 2.60218 | 6.36 |
| 34.5287 | 8604.75 | 0.0624 | 2.60196 | 6.53 |
| 35.2507 | 1734.20 | 0.1872 | 2.54399 | 1.32 |
| 35.9225 | 627.59 | 0.1560 | 2.49794 | 0.48 |
| 36.3727 | 1521.95 | 0.1560 | 2.46805 | 1.16 |
| 36.7591 | 10921.08 | 0.0624 | 2.44299 | 8.29 |
| 36.8558 | 16598.23 | 0.0780 | 2.43680 | 12.60 |
| 36.9072 | 15801.89 | 0.0624 | 2.43353 | 12.00 |
| 37.0273 | 9229.76 | 0.0780 | 2.43193 | 7.01 |
| 37.5024 | 915.96 | 0.1248 | 2.39626 | 0.70 |
| 37.6170 | 1026.81 | 0.1248 | 2.38922 | 0.78 |
| 37.9992 | 4085.21 | 0.1716 | 2.36606 | 3.10 |
| 38.3253 | 2144.23 | 0.1092 | 2.34668 | 1.63 |
| 38.7147 | 621.22 | 0.1560 | 2.32397 | 0.47 |
| 39.3994 | 2599.18 | 0.1404 | 2.28514 | 1.97 |
| 40.1021 | 988.71 | 0.1248 | 2.24670 | 0.75 |

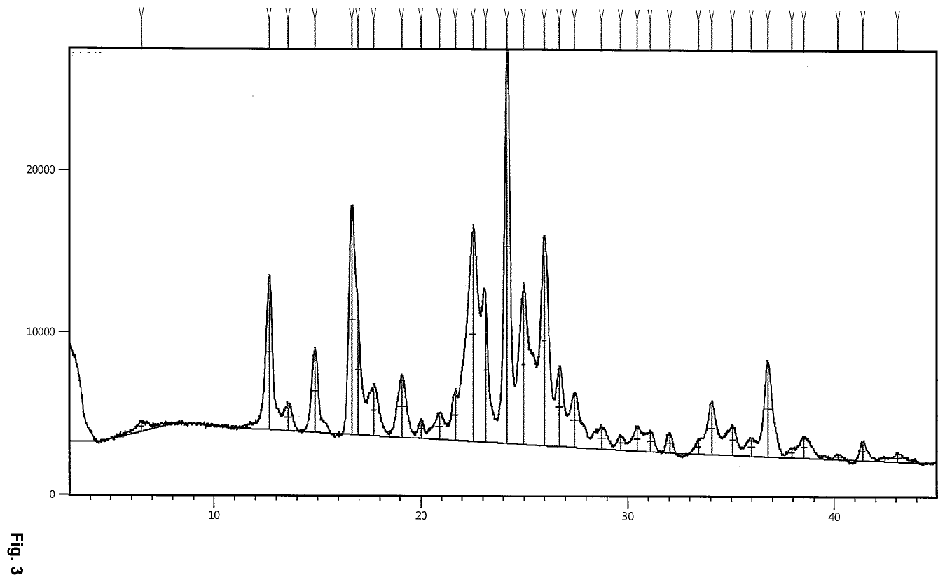

In a further preferred embodiment, the crystalline nicotinamide-β-D-ribofuranoside hydrogen malate used in step (A) of the method according to the first aspect is nicotinamide-β-D-ribofuranoside DL-hydrogen malate. This compound may be characterized by a powder X-ray diffraction pattern having peaks substantially as provided in Table 3, below, ±0.2 degrees two theta, or as provided in FIG. 3:

TABLE 3

| Pos. | Height | FWHM Left | d-spacing | Rel. Int. |
|---|---|---|---|---|
| [°2Th.] | [cts] | [°2Th.] | [Å] | [%] |
| 6.4916 | 602.91 | 0.2047 | 13.61608 | 2.47 |
| 12.6712 | 9566.63 | 0.1919 | 6.98619 | 39.22 |
| 13.5865 | 1644.76 | 0.3070 | 6.51749 | 6.74 |
| 14.8721 | 5163.66 | 0.3070 | 5.95686 | 21.17 |
| 16.6660 | 14179.37 | 0.2430 | 5.31953 | 58.13 |
| 16.9489 | 7986.14 | 0.1279 | 5.23135 | 32.74 |
| 17.7388 | 3121.02 | 0.1791 | 5.00015 | 12.79 |
| 19.0726 | 3887.84 | 0.3582 | 4.65339 | 15.94 |
| 20.0028 | 1196.35 | 0.2558 | 4.43904 | 4.90 |
| 20.8961 | 1618.71 | 0.3070 | 4.25124 | 6.64 |
| 21.6668 | 3108.24 | 0.1791 | 4.10174 | 12.74 |
| 22.5350 | 13238.45 | 0.2558 | 3.94563 | 54.27 |
| 23.1389 | 8854.70 | 0.1151 | 3.84401 | 36.30 |
| 24.1793 | 24392.79 | 0.2686 | 3.68090 | 100.00 |
| 24.9889 | 9891.47 | 0.1151 | 3.56347 | 40.55 |
| 25.9744 | 12996.05 | 0.1407 | 3.43045 | 53.28 |
| 26.7055 | 4918.49 | 0.2814 | 3.33817 | 20.16 |
| 27.4357 | 3377.82 | 0.3326 | 3.25097 | 13.85 |
| 28.7541 | 1330.72 | 0.3582 | 3.10484 | 5.46 |
| 29.6501 | 881.37 | 0.1791 | 3.01302 | 3.61 |
| 30.4640 | 1495.16 | 0.2558 | 2.93435 | 6.13 |
| 31.0878 | 1291.15 | 0.2303 | 2.87689 | 5.29 |
| 32.0173 | 1193.11 | 0.3070 | 2.79545 | 4.89 |
| 33.4314 | 911.84 | 0.2047 | 2.68037 | 3.74 |
| 34.0539 | 3246.16 | 0.2558 | 2.63279 | 13.31 |
| 35.0647 | 1874.40 | 0.2047 | 2.55917 | 7.68 |
| 35.9624 | 1143.74 | 0.3070 | 2.49733 | 4.69 |
| 36.7801 | 5938.21 | 0.3582 | 2.44366 | 24.34 |
| 37.9474 | 612.44 | 0.2047 | 2.37113 | 2.51 |
| 38.5227 | 1356.14 | 0.2558 | 2.33704 | 5.56 |
| 40.1563 | 339.33 | 0.2558 | 2.24566 | 1.39 |
| 41.3746 | 1129.63 | 0.2047 | 2.18231 | 4.63 |
| 43.0838 | 521.75 | 0.3070 | 2.09961 | 2.14 |

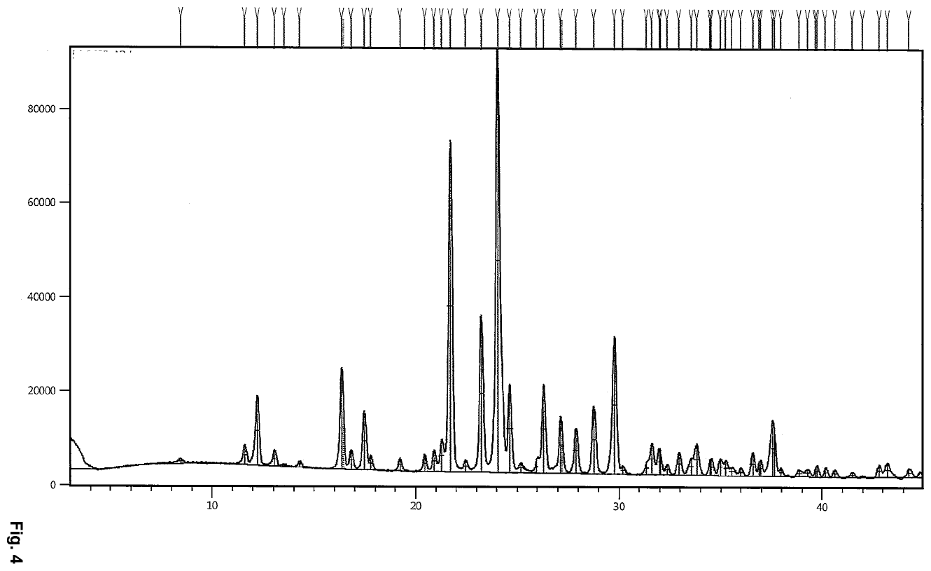

In a further particularly preferred embodiment, the crystalline nicotinamide-β-D-ribofuranoside hydrogen tartrate used in step (A) of the method according to the first aspect is nicotinamide-β-D-ribofuranoside D-hydrogen tartrate monohydrate. This compound may be characterized by a powder X-ray diffraction pattern having peaks substantially as provided in Table 4, below, ±0.2 degrees two theta, or as provided in FIG. 4:

TABLE 4

| Pos. | Height | FWHM Left | d-spacing | Rel. Int. |
|---|---|---|---|---|
| [°2Th.] | [cts] | [°2Th.] | [Å] | [%] |
| 8.4427 | 1105.85 | 0.1535 | 10.47327 | 1.22 |
| 11.5955 | 4172.99 | 0.1279 | 7.63174 | 4.61 |
| 12.2064 | 14705.94 | 0.1791 | 7.25111 | 16.24 |
| 13.0444 | 3289.98 | 0.0640 | 6.78714 | 3.63 |
| 13.5285 | 467.19 | 0.2047 | 6.54533 | 0.52 |
| 14.2848 | 1275.48 | 0.1535 | 6.20043 | 1.41 |
| 16.3545 | 21575.71 | 0.1404 | 5.41564 | 23.83 |
| 16.4347 | 15155.18 | 0.0780 | 5.40278 | 16.74 |
| 16.8125 | 4155.81 | 0.1560 | 5.26914 | 4.59 |
| 17.4631 | 12385.81 | 0.2028 | 5.07426 | 13.68 |
| 17.7789 | 3027.92 | 0.1872 | 4.98483 | 3.34 |
| 19.2171 | 2784.15 | 0.1560 | 4.61489 | 3.08 |
| 20.4255 | 3513.64 | 0.1872 | 4.34452 | 3.88 |
| 20.8956 | 4537.86 | 0.2028 | 4.24783 | 5.01 |
| 21.2720 | 6611.82 | 0.2028 | 4.17350 | 7.30 |
| 21.6994 | 70686.05 | 0.2652 | 4.09226 | 78.08 |
| 22.4487 | 2507.47 | 0.1560 | 3.95732 | 2.77 |
| 23.2293 | 33381.43 | 0.2184 | 3.82609 | 36.87 |
| 24.0319 | 90535.69 | 0.2184 | 3.70009 | 100.00 |
| 24.6286 | 18789.44 | 0.2496 | 3.61177 | 20.75 |
| 25.1774 | 1988.68 | 0.1872 | 3.53428 | 2.20 |
| 25.9477 | 2843.74 | 0.0936 | 3.43108 | 3.14 |
| 26.2952 | 18820.31 | 0.2496 | 3.38653 | 20.79 |
| 27.1345 | 12051.06 | 0.1560 | 3.28364 | 13.31 |
| 27.2135 | 8697.14 | 0.0780 | 3.28243 | 9.61 |
| 27.8905 | 9546.45 | 0.2496 | 3.19633 | 10.54 |
| 28.7630 | 14348.07 | 0.2184 | 3.10133 | 15.85 |
| 29.7764 | 29336.40 | 0.2496 | 2.99805 | 32.40 |
| 30.1789 | 1742.47 | 0.1560 | 2.95897 | 1.92 |
| 31.3589 | 2575.16 | 0.1404 | 2.85027 | 2.84 |
| 31.6114 | 6597.81 | 0.2028 | 2.82807 | 7.29 |
| 31.9725 | 5471.62 | 0.0624 | 2.79695 | 6.04 |
| 32.0262 | 4966.30 | 0.0936 | 2.79238 | 5.49 |
| 32.3674 | 2226.55 | 0.1248 | 2.76372 | 2.46 |
| 32.9500 | 4735.31 | 0.1872 | 2.71618 | 5.23 |
| 33.5558 | 3636.60 | 0.1560 | 2.66851 | 4.02 |
| 33.8129 | 6593.61 | 0.2496 | 2.64881 | 7.28 |
| 34.4780 | 3327.54 | 0.0780 | 2.59922 | 3.68 |
| 34.5356 | 3466.61 | 0.0936 | 2.59502 | 3.83 |
| 34.9811 | 3563.42 | 0.0780 | 2.56298 | 3.94 |
| 35.2429 | 3201.57 | 0.1560 | 2.54453 | 3.54 |
| 35.5541 | 1707.94 | 0.2184 | 2.52297 | 1.89 |
| 35.9811 | 1583.66 | 0.2184 | 2.49401 | 1.75 |
| 36.5723 | 4931.96 | 0.2340 | 2.45504 | 5.45 |
| 36.8813 | 2777.94 | 0.0780 | 2.43517 | 3.07 |
| 36.9482 | 3348.58 | 0.0936 | 2.43092 | 3.70 |
| 37.5567 | 11857.18 | 0.1248 | 2.39292 | 13.10 |
| 37.6362 | 10096.73 | 0.1404 | 2.38805 | 11.15 |
| 37.9557 | 1772.71 | 0.0936 | 2.36868 | 1.96 |
| 38.8483 | 1366.78 | 0.2496 | 2.31628 | 1.51 |

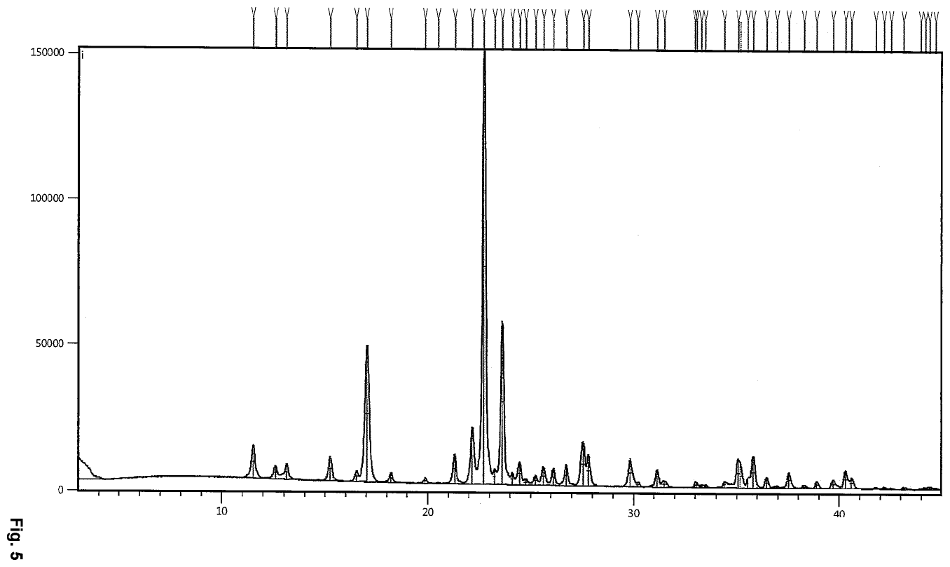

In a further preferred embodiment, the crystalline nicotinamide-β-D-ribofuranoside hydrogen tartrate used in step (A) of the method according to the first aspect is nicotinamide-β-L-ribofuranoside L-hydrogen tartrate. This compound may be characterized by a powder X-ray diffraction pattern having peaks substantially as provided in Table 5, below, ±0.2 degrees two theta, or as provided in FIG. 5:

TABLE 5

| Pos. | Height | FWHM Left | d-spacing | Rel. Int. |
|---|---|---|---|---|
| [°2Th.] | [cts] | [°2Th.] | [Å] | [%] |
| 11.5117 | 11200.84 | 0.1535 | 7.68714 | 7.48 |
| 12.5883 | 4412.06 | 0.1279 | 7.03201 | 2.95 |
| 13.1382 | 5170.21 | 0.1407 | 6.73885 | 3.45 |
| 15.2469 | 8205.64 | 0.1535 | 5.81130 | 5.48 |
| 16.5219 | 3562.59 | 0.1407 | 5.36559 | 2.38 |
| 17.0294 | 46534.70 | 0.2175 | 5.20680 | 31.08 |
| 18.1885 | 3338.98 | 0.1279 | 4.87754 | 2.23 |
| 19.8576 | 1936.08 | 0.0895 | 4.47116 | 1.29 |
| 20.4805 | 370.08 | 0.1279 | 4.33657 | 0.25 |
| 21.2805 | 10137.23 | 0.1279 | 4.17530 | 6.77 |
| 22.1515 | 19394.41 | 0.1535 | 4.01307 | 12.96 |
| 22.7119 | 149703.60 | 0.1663 | 3.91531 | 100.00 |
| 23.2457 | 5221.79 | 0.0512 | 3.82660 | 3.49 |
| 23.6134 | 55914.11 | 0.1663 | 3.76782 | 37.35 |
| 24.0980 | 4114.15 | 0.0895 | 3.69314 | 2.75 |
| 24.4411 | 7765.26 | 0.1407 | 3.64207 | 5.19 |
| 24.7474 | 2024.08 | 0.1151 | 3.59769 | 1.35 |
| 25.2253 | 3310.99 | 0.1279 | 3.53061 | 2.2 |
| 25.6075 | 6304.59 | 0.1663 | 3.47876 | 4.21 |
| 26.0932 | 5687.76 | 0.1407 | 3.41511 | 3.80 |
| 26.7150 | 7166.98 | 0.1535 | 3.33701 | 4.79 |
| 27.5250 | 15137.56 | 0.2047 | 3.24062 | 10.11 |
| 27.8002 | 10629.70 | 0.1407 | 3.20916 | 7.10 |
| 29.8120 | 9459.96 | 0.1407 | 2.99703 | 6.32 |
| 30.1918 | 1599.73 | 0.1023 | 2.96019 | 1.07 |
| 31.1237 | 5868.31 | 0.1535 | 2.87364 | 3.92 |
| 31.4626 | 2170.24 | 0.2303 | 2.84346 | 1.45 |
| 32.9642 | 1814.09 | 0.0780 | 2.71504 | 1.21 |
| 33.0404 | 1877.55 | 0.0640 | 2.71119 | 1.25 |
| 33.2757 | 1050.97 | 0.0768 | 2.69256 | 0.70 |
| 33.4753 | 934.01 | 0.1023 | 2.67696 | 0.62 |
| 34.3911 | 2124.57 | 0.1023 | 2.60774 | 1.42 |
| 35.0605 | 9970.35 | 0.1560 | 2.55736 | 6.66 |
| 35.1625 | 8673.63 | 0.0936 | 2.55651 | 5.79 |
| 35.5173 | 3085.28 | 0.0624 | 2.52551 | 2.06 |
| 35.7881 | 10815.35 | 0.1716 | 2.50701 | 7.22 |
| 36.4366 | 3605.79 | 0.1716 | 2.46386 | 2.41 |
| 36.9228 | 808.98 | 0.2496 | 2.43253 | 0.54 |
| 37.5169 | 5337.75 | 0.1560 | 2.39536 | 3.57 |
| 38.2736 | 1182.17 | 0.2184 | 2.34973 | 0.79 |
| 38.8883 | 2409.84 | 0.1716 | 2.31399 | 1.61 |
| 39.6760 | 3066.92 | 0.1404 | 2.26985 | 2.05 |
| 40.2830 | 6176.85 | 0.1716 | 2.23703 | 4.13 |
| 40.5831 | 3705.39 | 0.0936 | 2.22118 | 2.48 |
| 41.7493 | 674.67 | 0.1872 | 2.16179 | 0.45 |
| 42.1741 | 751.98 | 0.1872 | 2.14099 | 0.50 |
| 42.5125 | 407.16 | 0.1404 | 2.12473 | 0.27 |
| 43.1209 | 825.80 | 0.0780 | 2.09615 | 0.55 |
| 43.9559 | 380.32 | 0.0936 | 2.05825 | 0.25 |
| 44.1732 | 743.15 | 0.0936 | 2.04863 | 0.50 |

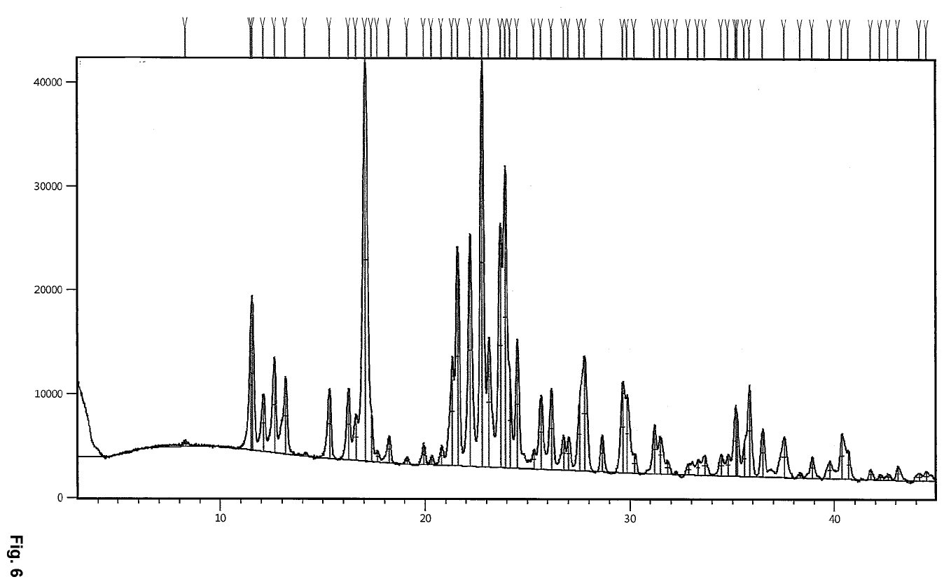

In a further preferred embodiment, the crystalline nicotinamide-β-D-ribofuranoside hydrogen tartrate used in step (A) of the method according to the first aspect is nicotinamide-β-D-ribofuranoside DL-hydrogen tartrate. This compound may be characterized by a powder X-ray diffraction pattern having peaks substantially as provided in Table 6, below, ±0.2 degrees two theta, or as provided in FIG. 6:

TABLE 6

| Pos. | Height | FWHM Left | d-spacing | Rel. Int. |
|---|---|---|---|---|
| [°2Th.] | [cts] | [°2Th.] | [Å] | [%] |
| 8.3072 | 551.28 | 0.2047 | 10.64375 | 1.39 |
| 11.4842 | 12590.46 | 0.0640 | 7.70547 | 31.84 |
| 11.5514 | 14904.11 | 0.1151 | 7.66075 | 37.70 |
| 12.0939 | 5535.53 | 0.1151 | 7.31835 | 14.00 |
| 12.6356 | 9270.40 | 0.1535 | 7.00577 | 23.45 |
| 13.1792 | 7450.29 | 0.1663 | 6.71800 | 18.84 |
| 14.1441 | 345.14 | 0.1535 | 6.26180 | 0.87 |
| 15.3154 | 6761.68 | 0.1535 | 5.78544 | 17.10 |
| 16.2510 | 6902.60 | 0.1663 | 5.45441 | 17.46 |
| 16.6164 | 4425.97 | 0.1407 | 5.33529 | 11.19 |
| 17.0694 | 38995.42 | 0.2047 | 5.19469 | 98.63 |
| 17.3813 | 4647.82 | 0.0512 | 5.10218 | 11.76 |
| 17.6574 | 1047.06 | 0.1023 | 5.02301 | 2.65 |
| 18.2347 | 2589.27 | 0.1407 | 4.86527 | 6.55 |
| 19.1062 | 673.09 | 0.1535 | 4.64527 | 1.70 |
| 19.9124 | 1996.71 | 0.1535 | 4.45898 | 5.05 |
| 20.3072 | 887.20 | 0.1279 | 4.37317 | 2.24 |
| 20.7939 | 1891.47 | 0.1535 | 4.27191 | 4.78 |
| 21.3241 | 10559.20 | 0.1535 | 4.16688 | 26.71 |
| 21.5806 | 21215.64 | 0.1663 | 4.11792 | 53.66 |
| 22.2002 | 22505.15 | 0.1919 | 4.00438 | 56.92 |
| 22.7919 | 39538.58 | 0.1919 | 3.90173 | 100.00 |
| 23.1194 | 12527.96 | 0.1535 | 3.84721 | 31.69 |
| 23.6904 | 23665.39 | 0.1535 | 3.75575 | 59.85 |
| 23.9145 | 29131.82 | 0.1279 | 3.72107 | 73.68 |
| 24.1548 | 9173.54 | 0.0895 | 3.68459 | 23.20 |
| 24.5068 | 12495.64 | 0.1791 | 3.63246 | 31.60 |
| 25.3161 | 1841.17. | 0.1279 | 3.51815 | 4.60 |
| 25.6584 | 7132.62 | 0.1407 | 3.47198 | 18.04 |
| 26.1650 | 7886.89 | 0.1663 | 3.40590 | 19.95 |
| 26.7609 | 3343.45 | 0.1023 | 3.33139 | 8.46 |
| 27.0106 | 3166.41 | 0.1023 | 3.30116 | 8.01 |
| 27.5367 | 7146.89 | 0.1407 | 3.23928 | 18.08 |
| 27.7875 | 11104.30 | 0.1535 | 3.21060 | 28.08 |
| 28.6467 | 3577.53 | 0.1535 | 3.11623 | 9.05 |
| 29.6650 | 8795.23 | 0.1279 | 3.01154 | 22.24 |
| 29.8675 | 7541.81 | 0.1023 | 2.99158 | 19.07 |
| 30.2186 | 1849.66 | 0.1279 | 2.95762 | 4.68 |
| 31.2001 | 4758.17 | 0.1791 | 2.86678 | 12.03 |
| 31.4762 | 3632.98 | 0.0768 | 2.84227 | 9.19 |
| 31.8369 | 1281.77 | 0.1279 | 2.81088 | 3.24 |
| 32.2436 | 301.91 | 0.1535 | 2.77635 | 0.76 |
| 32.8599 | 1056.56 | 0.1535 | 2.72567 | 2.67 |
| 33.3306 | 1490.86 | 0.1023 | 2.68825 | 3.7 |
| 33.6389 | 1842.81 | 0.1791 | 2.66431 | 4.66 |
| 34.4485 | 2040.78 | 0.1791 | 2.60353 | 5.16 |
| 34.7957 | 2063.81 | 0.1279 | 2.57834 | 5.22 |
| 35.1573 | 6826.29 | 0.0936 | 2.55053 | 17.26 |
| 35.2409 | 5684.59 | 0.0768 | 2.54678 | 14.38 |
| 35.5944 | 3446.74 | 0.0768 | 2.52229 | 8.72 |

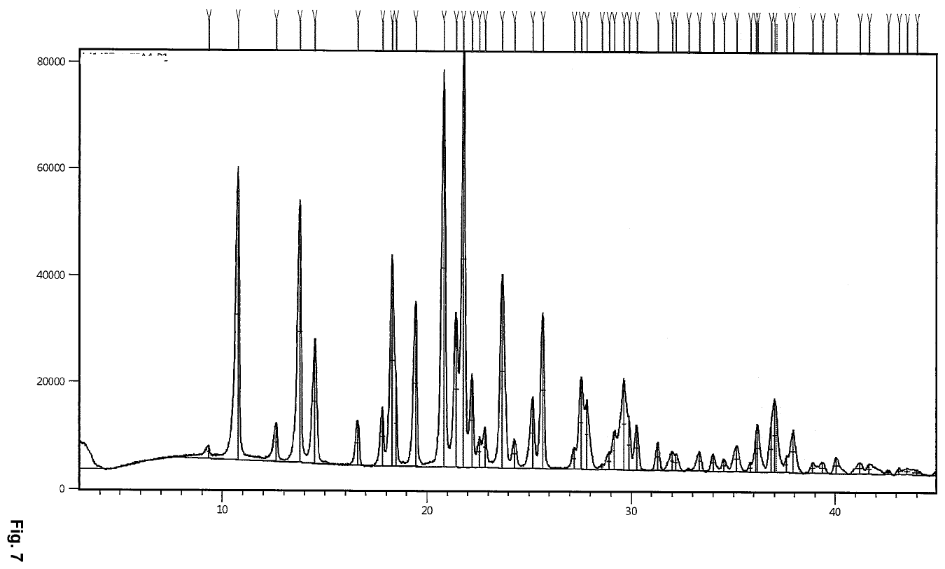

In a further preferred embodiment, the crystalline nicotinamide-2,3,5-O-triacetyl-β-D-ribofuranoside hydrogen tartrate used in step (A) of the method according to the second aspect is nicotinamide-2,3,5-triacetyl-O-β-D-ribofuranoside L-hydrogen tartrate. This compound may be characterized by a powder X-ray diffraction pattern having peaks substantially as provided in Table 7, below, ±0.2 degrees two theta, or as provided in FIG. 7:

TABLE 7

| Pos. | Height | FWHM Left | d-spacing | Rel. Int. |
|---|---|---|---|---|
| [°2Th.] | [cts] | [°2Th.] | [Å] | [%] |
| 9.3064 | 2347.46 | 0.0895 | 9.50315 | 3.00 |
| 10.7524 | 54988.21 | 0.2047 | 8.22821 | 70.17 |
| 12.6167 | 7227.54 | 0.1535 | 7.01622 | 9.22 |
| 13.7645 | 49185.18. | 0.2047 | 6.43363 | 62.76 |
| 14.5019 | 23374.03 | 0.2175 | 6.10811 | 29.83 |
| 16.5953 | 8289.38 | 0.2047 | 5.34201 | 10.58 |
| 17.7919 | 10891.17 | 0.1919 | 4.98534 | 13.90 |
| 18.2775 | 39457.79 | 0.2047 | 4.85397 | 50.35 |
| 18.4595 | 15797.39 | 0.0768 | 4.80652 | 20.16 |
| 19.4199 | 30898.77 | 0.1919 | 4.57093 | 39.43 |
| 20.7987 | 74506.22 | 0.2175 | 4.27092 | 95.08 |
| 21.4057 | 29048.75 | 0.1663 | 4.15117 | 37.07 |
| 21.7760 | 78364.38 | 0.2047 | 4.08141 | 100.00 |
| 22.1876 | 17507.69 | 0.1791 | 4.00662 | 22.34 |
| 22.5630 | 5652.40 | 0.1151 | 3.94079 | 7.21 |
| 22.8293 | 7556.27 | 0.1151 | 3.89543 | 9.64 |
| 23.6771 | 36209.59 | 0.1919 | 3.75783 | 46.21 |
| 24.2704 | 5293.40 | 0.1663 | 3.66730 | 6.75 |
| 25.1652 | 13110.92 | 0.1663 | 3.53890 | 16.73 |
| 25.6520 | 29248.77 | 0.1919 | 3.47283 | 37.32 |
| 27.2015 | 3937.84 | 0.1023 | 3.27842 | 5.03 |
| 27.5402 | 17275.90 | 0.1663 | 3.23887 | 22.05 |
| 27.8198 | 12918.63 | 0.0895 | 3.20695 | 16.49 |
| 28.5481 | 970.62 | 0.1279 | 3.12677 | 1.24 |
| 28.8967 | 3057.58 | 0.1279 | 3.08984 | 3.90 |
| 29.1513 | 7076.72 | 0.0640 | 3.06343 | 9.03 |
| 29.6275 | 17107.02 | 0.1407 | 3.01527 | 21.83 |
| 29.8910 | 9269.72 | 0.0895 | 2.98929 | 11.83 |
| 30.2515 | 8260.90 | 0.1535 | 2.95448 | 10.54 |
| 31.2838 | 5141.01 | 0.1919 | 2.85930 | 6.56 |
| 31.9853 | 3594.26 | 0.1023 | 2.79818 | 4.59 |
| 32.1680 | 3037.39 | 0.1791 | 2.78270 | 3.88 |
| 32.7773 | 523.69 | 0.1023 | 2.73235 | 0.67 |
| 33.3139 | 3622.17 | 0.0640 | 2.68956 | 4.62 |
| 33.9915 | 3211.57 | 0.1535 | 2.63748 | 4.10 |
| 34.5275 | 2365.82 | 0.1407 | 2.59776 | 3.02 |
| 35.1433 | 4786.90 | 0.1535 | 2.55363 | 6.11 |
| 35.8273 | 1682.02 | 0.1279 | 2.50643 | 2.15 |
| 36.0780 | 6948.34 | 0.0780 | 2.48753 | 8.87 |
| 36.1635 | 8783.18 | 0.1151 | 2.48390 | 11.21 |
| 36.8265 | 8675.69 | 0.1560 | 2.43867 | 11.07 |
| 37.0069 | 13561.59 | 0.1404 | 2.42720 | 17.31 |
| 37.0983 | 11547.67 | 0.1092 | 2.42744 | 14.74 |
| 37.5976 | 2995.69 | 0.0936 | 2.39041 | 3.82 |
| 37.9266 | 7992.80 | 0.2652 | 2.37042 | 10.20 |
| 38.8868 | 1967.52 | 0.1872 | 2.31408 | 2.51 |
| 39.3599 | 1961.34 | 0.3120 | 2.28734 | 2.50 |
| 40.0431 | 2967.56 | 0.2808 | 2.24988 | 3.79 |
| 41.2033 | 2016.08 | 0.3120 | 2.18917 | 2.57 |
| 41.6658 | 1798.38 | 0.1560 | 2.16593 | 2.29 |

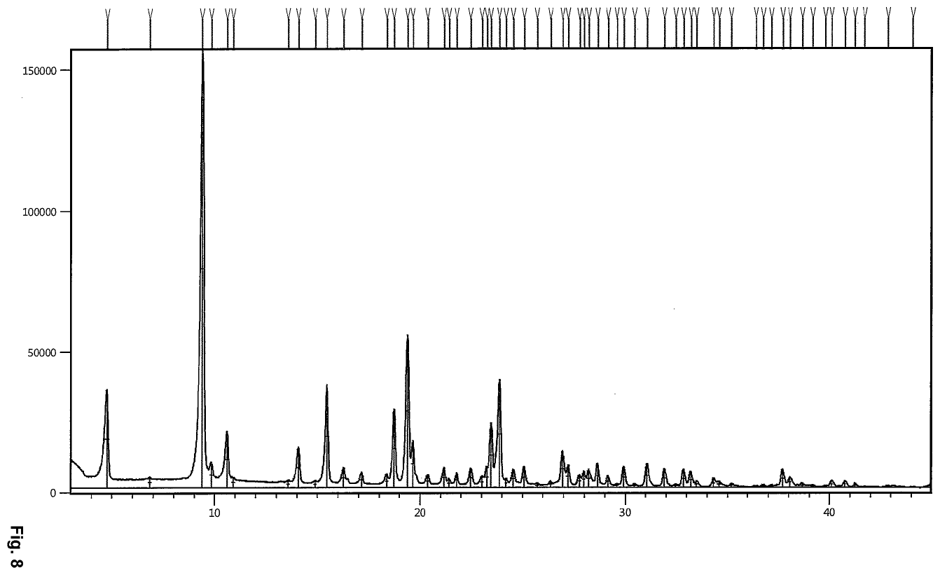

In a further preferred embodiment, the crystalline nicotinamide-2,3,5-O-triacetyl-β-D-ribofuranoside hydrogen tartrate used in step (A) of the method of the second aspect is nicotinamide-2,3,5-triacetyl-O-β-D-ribofuranoside D-hydrogen tartrate. This compound may be characterized by a powder X-ray diffraction pattern having peaks substantially as provided in Table 8, below, ±0.2 degrees two theta, or as in FIG. 8:

TABLE 8

| Pos. | Height | FWHM Left | d-spacing | Rel. Int. |
|---|---|---|---|---|
| [°2Th.] | [cts] | [°2Th.] | [Å] | [%] |
| 4.7520 | 34825.67 | 0.1279 | 18.59607 | 22.27 |
| 6.8248 | 3649.79 | 0.1023 | 12.95200 | 2.33 |
| 9.3927 | 156411.40 | 0.1407 | 9.41597 | 100.00 |
| 9.8311 | 8959.38 | 0.1151 | 8.99710 | 5.73 |
| 10.5898 | 20049.70 | 0.1279 | 8.35415 | 12.82 |
| 10.8918 | 3604.23. | 0.0768 | 8.12315 | 2.30 |
| 13.5669 | 2632.18 | 0.0768 | 6.52689 | 1.68 |
| 14.0604 | 14165.00 | 0.1535 | 6.29889 | 9.06 |
| 14.8805 | 2477.98 | 0.0768 | 5.95354 | 1.58 |
| 15.4419 | 36385.34 | 0.1407 | 5.73835 | 23.26 |
| 16.2548 | 6914.23 | 0.1023 | 5.45313 | 4.42 |
| 17.1363 | 5280.44 | 0.1535 | 5.17459 | 3.38 |
| 18.3514 | 4827.86 | 0.1023 | 4.83460 | 3.09 |
| 18.7218 | 27898.79 | 0.1407 | 4.73978 | 17.84 |
| 19.3655 | 53967.66 | 0.1535 | 4.58365 | 34.50 |
| 19.6270 | 16569.59 | 0.1023 | 4.52316 | 10.59 |
| 20.3529 | 4564.06 | 0.0895 | 4.36346 | 2.92 |
| 21.1360 | 6828.67 | 0.1279 | 4.20353 | 4.37 |
| 21.3815 | 2953.04 | 0.1023 | 4.15582 | 1.89 |
| 21.7618 | 5095.87 | 0.1535 | 4.08404 | 3.26 |
| 22.4599 | 6736.19 | 0.1279 | 3.95866 | 4.31 |
| 23.0021 | 3986.81 | 0.0895 | 3.86656 | 2.55 |
| 23.2351 | 7330.57 | 0.0768 | 3.82830 | 4.69 |
| 23.4473 | 22811.80 | 0.1279 | 3.79415 | 14.58 |
| 23.8524 | 38132.07 | 0.1535 | 3.73061 | 24.38 |
| 24.1716 | 2985.45 | 0.0512 | 3.68206 | 1.91 |
| 24.5306 | 6250.72 | 0.1407 | 3.62899 | 4.00 |
| 25.0542 | 7291.19 | 0.1279 | 3.55432 | 4.66 |
| 25.6757 | 1515.37 | 0.1023 | 3.46968 | 0.97 |
| 26.3310 | 2183.17 | 0.1151 | 3.38480 | 1.40 |
| 26.9259 | 12871.27 | 0.1535 | 3.31136 | 8.23 |
| 27.2043 | 7721.08 | 0.1279 | 3.27809 | 4.94 |
| 27.7527 | 4341.55 | 0.1023 | 3.21455 | 2.78 |
| 27.9615 | 5727.76 | 0.1023 | 3.19102 | 3.66 |
| 28.2032 | 6203.34 | 0.1151 | 3.16422 | 3.97 |
| 28.6321 | 8480.22 | 0.1151 | 3.11779 | 5.42 |
| 29.1374 | 4096.80 | 0.1151 | 3.06486 | 2.62 |
| 29.5708 | 1219.70 | 0.0768 | 3.02092 | 0.78 |
| 29.9108 | 7245.93 | 0.1151 | 2.98735 | 4.63 |
| 30.4399 | 1297.52 | 0.1151 | 2.93662 | 0.83 |
| 31.0508 | 8262.20 | 0.1407 | 2.88023 | 5.28 |
| 31.9092 | 6516.50 | 0.1407 | 2.80468 | 4.17 |
| 32.4553 | 1065.25 | 0.1279 | 2.75872 | 0.68 |
| 32.8369 | 6234.01 | 0.1151 | 2.72753 | 3.99 |
| 33.1800 | 5520.53 | 0.1279 | 2.70011 | 3.53 |
| 33.4767 | 2168.58 | 0.1023 | 2.67685 | 1.39 |
| 34.3025 | 3153.03 | 0.1151 | 2.61428 | 2.02 |
| 34.5793 | 2022.97 | 0.1407 | 2.59398 | 1.29 |
| 35.1728 | 1308.53 | 0.0895 | 2.55156 | 0.84 |
| 36.3834 | 468.74 | 0.2047 | 2.46939 | 0.30 |

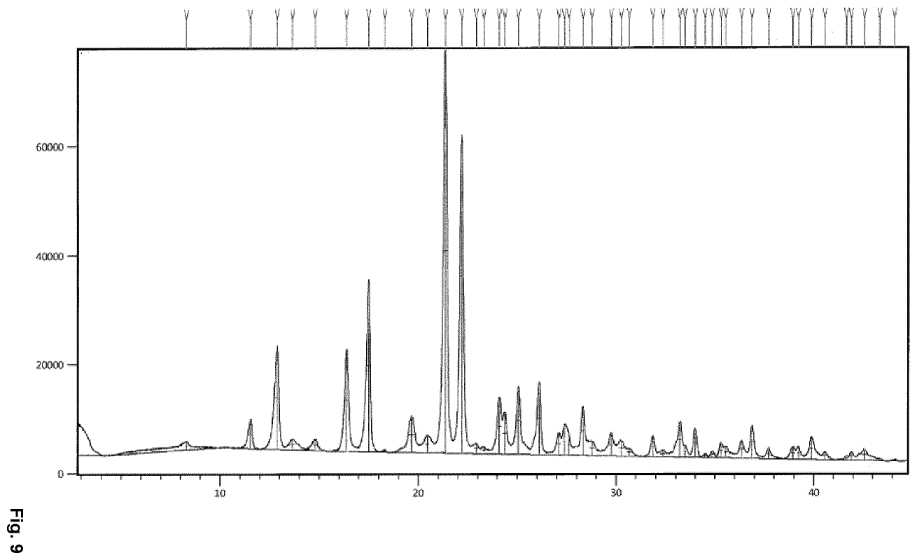

In a further preferred embodiment, the crystalline nicotinamide-β-D-ribofuranoside hydrogen tartrate used in step (A) of the method according to the first aspect is anhydrous nicotinamide-β-D-ribofuranoside D-hydrogen tartrate. This compound may be characterized by a powder X-ray diffraction pattern having peaks substantially as provided in Table 9, below, ±0.2 degrees two theta, or as in FIG. 9:

TABLE 9

| Pos. | Height | FWHM Left | d-spacing | Rel. Int. |
|---|---|---|---|---|
| [°2Th.] | [cts] | [°2Th.] | [Å] | [%] |
| 8.2867 | 1565.03 | 0.1279 | 10.67015 | 2.10 |
| 11.5441 | 5477.05 | 0.1663 | 7.66563 | 7.34 |
| 12.8809 | 19026.31 | 0.1919 | 6.87292 | 25.50 |
| 13.6583 | 2145.15 | 0.2047 | 6.48342 | 2.87 |
| 14.7982 | 2238.27 | 0.2047 | 5.98644 | 3.00 |
| 16.3923 | 18852.04 | 0.1663 | 5.40772 | 25.26 |
| 17.4939 | 31734.04 | 0.1791 | 5.06958 | 42.53 |
| 18.2961 | 447.64 | 0.1279 | 4.84910 | 0.60 |
| 19.6742 | 6616.60 | 0.2814 | 4.51243 | 8.87 |
| 20.4519 | 3327.08 | 0.1535 | 4.34256 | 4.46 |
| 21.3667 | 74623.20 | 0.1919 | 4.15865 | 100.00 |
| 22.2026 | 58527.36 | 0.1919 | 4.00395 | 78.43 |
| 22.9236 | 1946.12 | 0.1535 | 3.87962 | 2.61 |
| 23.3038 | 1293.31 | 0.1023 | 3.81718 | 1.73 |
| 24.1005 | 10296.35 | 0.1535 | 3.69276 | 13.80 |
| 24.3711 | 7580.65 | 0.1535 | 3.65237 | 10.16 |
| 25.0633 | 12509.80 | 0.1535 | 3.55305 | 16.76 |
| 26.1075 | 13375.49 | 0.1663 | 3.41327 | 17.92 |
| 27.1046 | 4134.24 | 0.1535 | 3.28992 | 5.54 |
| 27.3697 | 5451.44 | 0.1023 | 3.25865 | 7.31 |
| 27.6019 | 4053.73 | 0.1023 | 3.23177 | 5.43 |
| 28.3123 | 9045.58 | 0.1535 | 3.15228 | 12.12 |
| 28.7576 | 2770.85 | 0.1791 | 3.10447 | 3.71 |
| 29.7420 | 4272.59 | 0.1151 | 3.00392 | 5.73 |
| 30.2505 | 2903.11 | 0.2303 | 2.95457 | 3.89 |
| 30.6625 | 1476.96 | 0.1791 | 2.91581 | 1.98 |
| 31.8526 | 3847.53 | 0.1151 | 2.80953 | 5.16 |
| 32.3522 | 1209.36 | 0.2047 | 2.76727 | 1.62 |
| 33.2255 | 6634.29 | 0.1407 | 2.69651 | 8.89 |
| 33.4595 | 2216.67 | 0.1023 | 2.67819 | 2.97 |
| 33.9924 | 5402.04 | 0.1279 | 2.63741 | 7.24 |
| 34.5081 | 725.11 | 0.1279 | 2.59917 | 0.97 |
| 34.8645 | 1193.46 | 0.1791 | 2.57341 | 1.60 |
| 35.3079 | 2701.51 | 0.1535 | 2.54210 | 3.62 |
| 35.5426 | 2210.45 | 0.1279 | 2.52586 | 2.96 |
| 36.3433 | 3310.00 | 0.1407 | 2.47202 | 4.44 |
| 36.8835 | 6123.57 | 0.1535 | 2.43705 | 8.21 |
| 37.7104 | 1917.12 | 0.1023 | 2.38549 | 2.57 |
| 38.9440 | 2230.62 | 0.1279 | 2.31272 | 2.99 |
| 39.2338 | 2324.44 | 0.0768 | 2.29630 | 3.11 |
| 39.8924 | 4022.79 | 0.1151 | 2.25990 | 5.39 |
| 40.5627 | 1485.94 | 0.1791 | 2.2409 | 1.99 |
| 41.6641 | 814.42 | 0.1023 | 2.16781 | 1.09 |
| 41.9184 | 1443.54 | 0.1535 | 2.15525 | 1.93 |
| 42.5626 | 2097.08 | 0.1791 | 2.12410 | 2.81 |
| 43.3574 | 415.83 | 0.1535 | 2.08699 | 0.56 |
| 44.0950 | 235.65 | 0.3070 | 2.05378 | 0.32 |

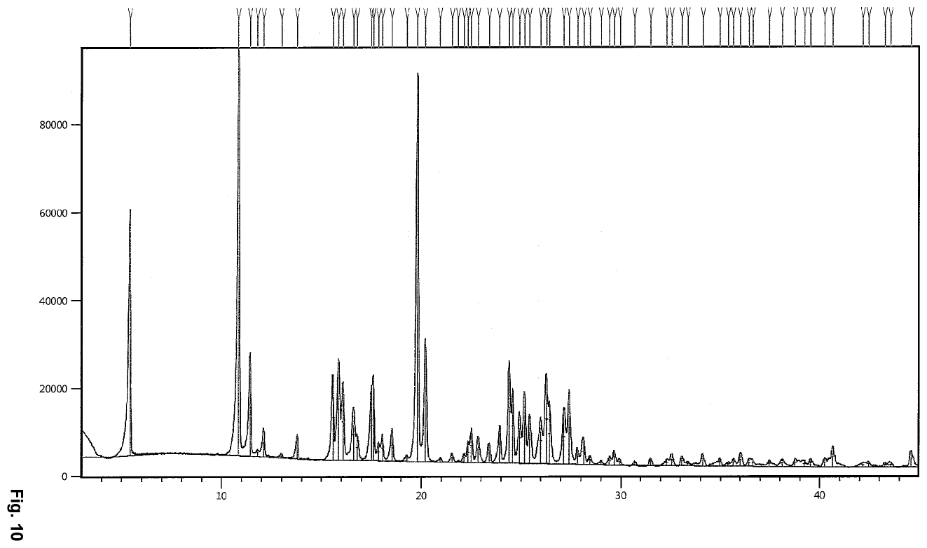

In a further preferred embodiment, the crystalline nicotinamide-β-D-ribofuranoside salt obtained in the methods according to the invention is crystalline nicotinamide-β-D-ribofuranoside tosylate. This compound may be characterized by a powder X-ray diffraction pattern having peaks substantially as provided in Table 10, below, ±0.2 degrees two theta, or as provided in FIG. 10:

TABLE 10

| Pos. | Height | FWHM Left | d-spacing | Rel. Int. |
|---|---|---|---|---|
| [°2Th.] | [cts] | [°2Th.] | [Å] | [%] |
| 5.4114 | 55141.85 | 0.0640 | 16.33132 | 60.69 |
| 10.8638 | 90855.91 | 0.0768 | 8.14402 | 100.00 |
| 11.4355 | 23106.79 | 0.0768 | 7.73814 | 25.43 |
| 11.8020 | 1709.69 | 0.0640 | 7.49864 | 1.88 |
| 12.1134 | 6517.56 | 0.0768 | 7.30661 | 7.17 |
| 13.0059 | 1051.84 | 0.0895 | 6.80713 | 1.16 |
| 13.7917 | 5602.61 | 0.0768 | 6.42100 | 6.17 |
| 15.5682 | 19380.94 | 0.0895 | 5.69207 | 21.33 |
| 15.8570 | 23042.82 | 0.1023 | 5.58905 | 25.36 |
| 16.0860 | 17526.41 | 0.0895 | 5.51000 | 19.29 |
| 16.6231 | 11972.53 | 0.1151 | 5.33314 | 13.18 |
| 16.7855 | 5862.36 | 0.1151 | 5.28192 | 6.45 |
| 17.4918 | 15906.82 | 0.0895 | 5.07021 | 17.51 |
| 17.6001 | 18916.75 | 0.0640 | 5.03925 | 20.82 |
| 17.8628 | 4208.95 | 0.1023 | 4.96573 | 4.63 |
| 18.0491 | 5954.49 | 0.0895 | 4.91490 | 6.55 |
| 18.5388 | 7234.88 | 0.1279 | 4.78614 | 7.96 |
| 19.2749 | 1482.49 | 0.0768 | 4.60499 | 1.63 |
| 19.8085 | 88196.89 | 0.1151 | 4.48213 | 97.07 |
| 20.2090 | 27477.48 | 0.1151 | 4.39420 | 30.24 |
| 20.9429 | 974.36 | 0.1151 | 4.24186 | 1.07 |
| 21.5493 | 2060.54 | 0.0895 | 4.12383 | 2.27 |
| 21.8408 | 410.90 | 0.0768 | 4.06945 | 0.45 |
| 22.1440 | 1974.54 | 0.0512 | 4.01442 | 2.17 |
| 22.3491 | 4642.83 | 0.0640 | 3.97803 | 5.11 |
| 22.5116 | 7879.22 | 0.0895 | 3.94968 | 8.67 |
| 22.8527 | 5966.86 | 0.1151 | 3.89150 | 6.57 |
| 23.4090 | 4400.97 | 0.1023 | 3.80025 | 4.84 |
| 23.9297 | 8473.44 | 0.1023 | 3.71873 | 9.33 |
| 24.4005 | 23352.15 | 0.0895 | 3.64804 | 25.70 |
| 24.5863 | 16985.00 | 0.0640 | 3.62089 | 18.69 |
| 24.9164 | 11631.20 | 0.1023 | 3.57366 | 12.80 |
| 25.1743 | 16205.54 | 0.0895 | 3.53763 | 17.84 |
| 25.4358 | 10999.88 | 0.1279 | 3.50186 | 12.11 |
| 25.9827 | 10517.93 | 0.1023 | 3.42938 | 11.58 |
| 26.2670 | 20560.25 | 0.1023 | 3.39289 | 22.63 |
| 26.4312 | 13771.82 | 0.1023 | 3.37219 | 15.16 |
| 27.1253 | 12379.26 | 0.1407 | 3.28746 | 13.63 |
| 27.4170 | 16944.38 | 0.1023 | 3.25314 | 18.65 |
| 27.8272 | 3806.18 | 0.0768 | 3.20611 | 4.19 |
| 28.1295 | 6035.88 | 0.1279 | 3.17234 | 6.64 |
| 28.4475 | 2072.70 | 0.1023 | 3.13760 | 2.28 |
| 29.0005 | 1007.63 | 0.1023 | 3.07902 | 1.11 |
| 29.4328 | 1989.98 | 0.0895 | 3.03477 | 2.19 |
| 29.6777 | 3312.50 | 0.0768 | 3.01029 | 3.65 |
| 29.9529 | 1358.92 | 0.1023 | 2.98325 | 1.50 |
| 30.6951 | 978.74 | 0.0895 | 2.91278 | 1.08 |
| 31.4864 | 1678.36 | 0.0895 | 2.84137 | 1.85 |
| 32.3077 | 1443.47 | 0.0895 | 2.77099 | 1.59 |
| 32.5601 | 2749.63 | 0.1023 | 2.75008 | 3.03 |

### Salt Metathesis of Nicotinamide-β-D-Ribofuranoside Salts, Respectively Nicotinamide-2,3,5-Tri-O-Acyl-β-D-Ribofuranoside Salts using an Acid for Counter-Ion Exchange

The inventors of the present invention have further discovered that the above reaction scheme

NR⁺X⁻+Cat⁺Y⁻→NR⁺Y⁻+Cat⁺X⁻

AcONR⁺X⁻+Cat⁺Y⁻→AcONR⁺Y⁻+Cat⁺X⁻,

when Cat⁺ is H⁺ and H⁺Y⁻ is a stronger acid than malic acid or tartaric acid, may be generalized to further salts of nicotinamide-p-D-ribofuranoside salts NR⁺X⁻, respectively nicotinamide-2,3,5-tri-O-acyl-β-D-ribofuranoside salt AcONR⁺X⁻ in order to prepare NR⁺X⁻ salts.

Accordingly, in another aspect, the invention relates to a method of making a nicotinamide-β-D-ribofuranoside salt NR⁺Y⁻ from a nicotinamide-β-D-ribofuranoside salt NR⁺X⁻, comprising step (A):
(A) subjecting the nicotinamide-β-D-ribofuranoside salt NR⁺X to an acid H⁺Y⁻ to afford the nicotinamide-β-D-ribofuranoside salt NR⁺Y⁻ and H⁺X⁻, wherein pKₐ H⁺Y⁻<pKₐ H⁺X⁻.

In a subsequent step, the nicotinamide-p-D-ribofuranoside salt NR⁺Y⁻ may be isolated according to known methods.

The reaction according to step (A) may be further supported if NR⁺Y⁻ is less soluble in the used solvent than NR⁺X.

The reaction may also be performed using a nicotinamide-2,3,5-tri-O-acyl-β-D-ribofuranoside salt AcONR⁺X⁻ as starting material, wherein simultaneously deacylation takes place.

Thus, according to a further aspect, the invention relates to a method of making a nicotinamide-β-D-ribofuranoside salt NR⁺Y⁻ from a nicotinamide-2,3,5-tri-O-acyl-β-D-ribofuranoside salt AcONR⁺X⁻, comprising step (A):
(A) subjecting the nicotinamide-2,3,5-tri-O-acyl-β-D-ribofuranoside salt AcONR⁺X⁻ to an acid H⁺Y⁻ to afford the nicotinamide-p-D-ribofuranoside salt NR⁺Y⁻ and H⁺X⁻, wherein pKₐ H⁺Y⁻<pKₐ H⁺X⁻.

In a subsequent step, the nicotinamide-β-D-ribofuranoside salt NR⁺Y⁻ may be isolated according to known methods.

Acyl in AcONR⁺X has the definition as specified above, i.e. acyl is independently selected from alkyl carbonyl, aryl carbonyl and heteroaryl carbonyl, preferably from C₁₋₁₀ alkyl carbonyl and benzoyl, and is more preferably acetyl, and wherein acyl is optionally independently substituted with one or more substituents selected from: C₁₋₆ alkyl, C₁₋₆ alkoxy, C₁₋₆ thioalkyl, halogen, nitro, cyano, NH(C₁₋₆ alkyl), N(C₁₋₆ alkyl)₂, and SO₂N(C₁₋₆ alkyl)₂.

As disclosed above, the reaction preferably is carried out in an alcohol selected from the group consisting of methanol, ethanol, propanol (e.g., n-propanol, iso-propanol), or butanol (e.g., n-butanol, iso-butanol, sec-butanol, tert.-butanol), or a mixture of two or more thereof, optionally wherein the alcohol or the mixture of alcohol comprises water.

Preferably, in step (A) a suspension of the nicotinamide-β-D-ribofuranoside salt or nicotinamide-2,3,5-tri-O-acyl-β-D-ribofuranoside salt in one or more of the alcohols defined above, optionally comprising water, and a suitable acid are combined with one another to carry out step (A), i.e. the nicotinamide-β-D-ribofuranoside salt is formed by counter-ion exchange and typically precipitates so that it can be isolated, for example, by filtration.

Preferably, the acid H⁺Y⁻ is used in a molar excess compared to the starting material nicotinamide-β-D-ribofuranoside salt NR⁺X or nicotinamide-2,3,5-tri-O-acyl-β-D-ribofuranoside salt AcONR⁺X⁻. Preferably, more than 1.1 molar equivalents of acid H⁺Y⁻ are used, further preferred at least 1.2 or 1.3 or 1.4 or 1.5 equivalents.

Exemplarily mentioned is the preparation of nicotinamide-β-D-ribofuranoside tosylate starting from nicotinamide-2,3,5-tri-O-acetyl-β-D-ribofuranoside triflate upon subjecting same to p-toluenesulfonic acid (pKₐ=−2.8) wherein triflic acid (pKₐ=0.23) is formed.

Further exemplarily mentioned is the preparation of nicotinamide-β-D-ribofuranoside chloride or nicotinamide-β-D-ribofuranoside bromide starting from nicotinamide-β-D-ribofuranoside tosylate upon subjecting same to hydrochloric acid (pKₐ=−6) or hydrobromic acid (pKₐ=−8.9) wherein p-toluenesulfonic acid (pKₐ=−2.8) is formed.

### Salt Metathesis of Nicotinamide-β-D-Ribofuranoside Hydrogen Malate or Tartrate, Respectively Nicotinamide-2,3,5-Tri-O-Acyl-β-D-Ribofuranoside Hydrogen Malate or Tartrate, Using More Than One Counter-Ion for Counter-Ion Exchange

In particular embodiments of the first aspect or the second aspect, the invention discloses methods, wherein in the counter-ion exchange according to step (A) more than one counter-ion is employed.

Preferably, in one embodiment of the first aspect, in the counter-ion exchange according to step (A) more than one counter-ion is employed.

In one embodiment, two counter-ions are employed.

In a particularly preferred embodiment, the counter-ions are selected from chloride and iodide.

The inventors surprisingly discovered that in the resulting crystalline nicotinamide-β-D-ribofuranoside salt chloride and iodide are co-crystallized.

The term “co-crystallization” as used in this disclosure means that more than one counter-ion is incorporated in the crystal lattice of the formed nicotinamide-β-D-ribofuranoside salt.

Further surprisingly, the inventors of the present invention discovered that depending on the ratio of chloride to iodide used in the counter-ion exchange reaction according to step (A), different ratios of chloride and iodide can be set in the resulting co-crystallized nicotinamide-β-D-ribofuranoside salt.

Specifically, the present invention discloses co-crystallized nicotinamide-β-D-ribofuranoside (chloride/iodide) salts, wherein the molar ratio of chloride to iodide is 5:1, 3:1, 2:1, 1.5:1 and 1:1.

The term “ratio of chloride to iodide” as termed herein, means e.g. for a ratio of chloride to iodide of 2 : 1 that in the crystal lattice of nicotinamide-β-D-ribofuranoside chloride every third chloride is replaced by iodide. Thus, the ratio is a numerical ratio in terms of the molar ratio.

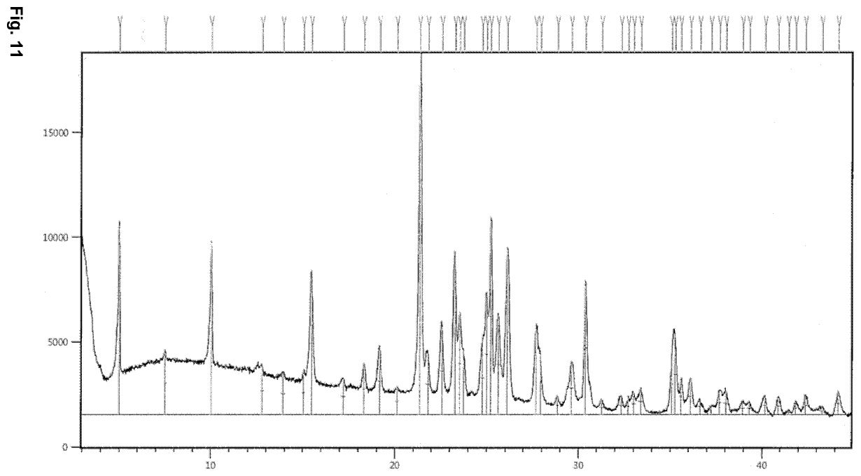

Exemplarily characterized is a co-crystallized nicotinamide-β-D-ribofuranoside (chloride/iodide), wherein choride and iodide are present in a ratio of 2:1, by a powder X-ray diffraction pattern having peaks substantially as provided in Table 11, below, ±0.2 degrees two theta, or as provided in FIG. 11:

TABLE 11

| Pos. | Height | FWHM Left | d-spacing | Rel. Int. |
|---|---|---|---|---|
| [°2Th.] | [cts] | [°2Th.] | [Å] | [%] |
| 5.0145 | 9009.81 | 0.0640 | 17.62320 | 52.52 |
| 7.5053 | 3011.24 | 0.1023 | 11.77919 | 17.55 |
| 10.0265 | 8180.03 | 0.0640 | 8.82222 | 47.68 |
| 12.7916 | 2284.39 | 0.1279 | 6.92070 | 13.32 |
| 13.9279 | 1975.21 | 0.2047 | 6.35851 | 11.51 |
| 15.0558 | 2102.96 | 0.0768 | 5.88463 | 12.26 |
| 15.4737 | 6813.15 | 0.1407 | 5.72662 | 39.71 |
| 17.2093 | 1673.21 | 0.2047 | 5.15279 | 9.75 |
| 18.3247 | 2438.66 | 0.0895 | 4.84159 | 14.21 |
| 19.1959 | 3191.11 | 0.1279 | 4.62376 | 18.60 |
| 20.1239 | 1232.77 | 0.1535 | 4.41259 | 7.19 |
| 21.3699 | 17156.45 | 0.1279 | 4.15804 | 100.00 |
| 21.8183 | 2916.97 | 0.1791 | 4.07359 | 17.00 |
| 22.5768 | 4141.68 | 0.1535 | 3.93842 | 24.14 |
| 23.2749 | 7730.58 | 0.1407 | 3.82186 | 45.06 |
| 23.5412 | 4873.80 | 0.1023 | 3.77922 | 28.41 |
| 23.7556 | 3096.08 | 0.0640 | 3.74560 | 18.05 |
| 24.7659 | 3460.88 | 0.1535 | 3.59504 | 20.17 |
| 24.9895 | 5798.73 | 0.0895 | 3.56338 | 33.80 |
| 25.2316 | 9408.73 | 0.1023 | 3.52974 | 54.84 |
| 25.6281 | 4771.62 | 0.1407 | 3.47602 | 27.81 |
| 26.1168 | 7852.33 | 0.1791 | 3.41207 | 45.77 |
| 27.6892 | 4015.88 | 0.0640 | 3.22178 | 23.41 |
| 27.9247 | 3002.17 | 0.0512 | 3.19514 | 17.50 |
| 28.8784 | 853.59 | 0.1023 | 3.09176 | 4.98 |
| 29.6355 | 2531.47 | 0.1791 | 3.01447 | 14.76 |
| 30.3781 | 6364.44 | 0.0895 | 2.94245 | 37.10 |
| 31.2642 | 697.21 | 0.1791 | 2.86105 | 4.06 |
| 32.3227 | 878.89 | 0.1791 | 2.76974 | 5.12 |
| 32.7107 | 812.23 | 0.0768 | 2.73776 | 4.73 |
| 33.0002 | 1013.47 | 0.1279 | 2.71441 | 5.91 |
| 33.3946 | 1227.54 | 0.1791 | 2.68324 | 7.15 |
| 35.0887 | 2850.83 | 0.0936 | 2.55537 | 16.62 |
| 35.2597 | 3814.90 | 0.0895 | 2.54547 | 22.24 |
| 35.6010 | 1706.69 | 0.0895 | 2.52184 | 9.95 |
| 36.1084 | 1704.02 | 0.1791 | 2.48756 | 9.93 |
| 36.6149 | 755.62 | 0.1535 | 2.45431 | 4.40 |
| 37.2392 | 384.25 | 0.1535 | 2.41458 | 2.24 |
| 37.6964 | 1118.13 | 0.1535 | 2.38634 | 6.52 |
| 38.0429 | 1102.17 | 0.1791 | 2.36540 | 6.42 |
| 38.9514 | 588.72 | 0.2047 | 2.31230 | 3.43 |
| 39.3413 | 574.10 | 0.1791 | 2.29028 | 3.35 |
| 40.1838 | 865.82 | 0.1279 | 2.24418 | 5.05 |
| 40.9033 | 819.66 | 0.2303 | 2.20635 | 4.78 |
| 41.4594 | 184.12 | 0.1535 | 2.17804 | 1.07 |
| 41.8473 | 602.54 | 0.1279 | 2.15874 | 3.51 |
| 42.3788 | 903.63 | 0.1535 | 2.13289 | 5.27 |
| 43.2887 | 370.36 | 0.2558 | 2.09014 | 2.16 |
| 44.1889 | 1051.11 | 0.1023 | 2.04963 | 6.13 |

Accordingly, the present invention also relates to a crystalline form of co-crystallized nicotinamide-β-D-ribofuranoside salt, wherein the anions of the salt comprise or consist of chloride and iodide.

In embodiments, the molar ratio of chloride to iodide is 5:1, 3:1, 2:1, 1.5:1 and 1:1. The X-ray diffraction patterns of respective crystals are very similar and differ only in the intensity of individual peaks.

In one embodiment, the invention relates to co-crystallized nicotinamide-p-D-ribofuranoside (chloride, iodide) characterized by a powder X-ray diffraction pattern having peaks substantially as provided in Table 11, ±0.2 degrees two theta, or as provided in FIG. 11.

The co-crystallized nicotinamide-β-D-ribofuranoside (chloride, iodide) may be used in a nutritional supplement of a pharmaceutical composition.

Accordingly, the invention also relates to a nutritional supplement or a pharmaceutical composition comprising the co-crystallized nicotinamide-β-D-ribofuranoside (chloride, iodide).

The following Examples further illustrate the present invention.

### EXAMPLES

### Preparation of Starting Materials

### Example 1: Preparation of Nicotinamide-β-D-Ribofuranoside L-Hydrogen Tartrate and Nicotinamide-β-D-Ribofuranoside DL-Hydrogen Tartrate from Nicotinamide-β-D-Ribofuranoside Bromide

### Example 1a: Nicotinamide-β-D-Ribofuranoside L-Hydrogen Tartrate

3.90 g of L-tartaric acid (26.0 mmol) were dissolved in 10 ml methanol with stirring. The colorless solution was cooled in an ice bath and 3.64 ml triethylamine (26.1 mmol) added. The pH of the slightly yellowish solution was around 4-4.5. In this manner 15 ml of a 1.73 molar solution of TEAL-hydrogen tartrate (TEA=triethyl amine) was prepared.

5.8 g nicotinamide-β-D-ribofuranoside bromide (NR·Br) were dissolved with stirring in 3.5 ml water at room temperature. 10 ml methanol were added. 10 ml of the above prepared solution of triethylammonium L-hydrogen tartrate were added to the clear colorless solution. White product starts precipitating.

The suspension was stirred for a further hour at room temperature. The product was filtered, washed with methanol and dried in vacuum at 35° C. 6.62 g (95%) of a white, crystalline powder were obtained; mp: 129-130° C.; IC: Residual bromide 0.20%. The solid may be recrystallized from aqueous methanol, if desired.

¹H-NMR (400 MHz, D₂O): 3.82 (dd, 1H, H5′), 3.97 (dd, 1H, H5′), 4.28 (t, 1H, H3′), 4.38-4.45 (m, 2H, H4′, H2′), 4.41 (s, 2H, 2x CHOH, H-tartrate), 6.17 (d, 1H, H1′), 8.20 (t, 1H, H5), 8.90 (d, 1H, H4), 9.19 (d, 1H, H6), 9.52 (s, 1H, H2). Impurities: <1 mol % nicotinamide; 1.2 mol % TEA salt: 1.19 (t, 9H), 3.11 (q, 6H). Solvents: 7.3 mol % methanol: 3.25 (s, 3H).

¹³C-NMR (100 MHz, D₂O): 60.2 (C5′), 69.7 (C3′), 72.8 (2x CHOH, H-tartrate), 77.4 (C2′), 87.6 (C4′), 99.9 (C1′), 128.4 (C5), 133.9 (C3), 140.4 (C2), 142.6 (C6), 145.6 (C4), 165.8 (CONH2), 176.3 (2x COO, H-tartrate). Impurity: 8.2, 46.6 (TEA). Solvents: 48.9 (methanol).

XRD: crystalline (see FIG. 5)

### Example 1 b: Nicotinamide-β-D-Ribofuranoside DL-Hydrogen Tartrate

The following crystalline nicotinamide-β-D-ribofuranoside salt of the table was prepared analogously to the method described above:

| | Yield | Mp | Residual Bromide |
|---|---|---|---|
| Anion | [%] | [° C.] | (IC) [%] |
| DL-hydrogen | 90 | 112-114 | 0.1 |
| tartrate (FIG. 6) | | | |

### Example 2: Preparation of Nicotinamide-β-D-Ribofuranoside L-Hydrogen Malate, D-Hydrogen Malate, Nicotinamide-β-D-Ribofuranoside DL-Hydrogen Malate and D-hydrogen tartrate from nicotinamide-β-D-ribofuranoside bromide

### Example 2a: Nicotinamide-β-D-Ribofuranoside L-Hydrogen Malate

5.8 g nicotinamide-β-D-ribofuranoside bromide were suspended in 10 ml methanol upon stirring. 10 ml of a 1.73 molar solution of triethylammonium L- hydrogen malate were added. The suspension was heated until the solids dissolved completely. After cooling, a white solid precipitated. The suspension was stirred for 30 min and then filtered. The residue was washed with methanol and dried in vacuo at 35° C. 4.15 g (62%) of a white crystalline powder was obtained. Mp: 116.5-117° C. IC: Residual bromide 0.10%. The product may be recrystallized from methanol, if desired.

¹H-NMR (400 MHz, D₂O): 2.53 (dd, 1H, CH₂, H-malate), 2.72 (dd, 1H, CH₂, H-malate), 3.81 (dd, 1H, H5′), 3.97 (dd, 1H, H5′), 4.28 (t, 1H, H3′), 4.29 (dd, 1H, CHOH, H-malate), 4.38-4.45 (m, 2H, H4′, H2′), 6.17 (d, 1H, H1′), 8.20 (t, 1H, H5), 8.90 (d, 1H, H4), 9.19 (d, 1H, H6), 9.52 (s, 1H, H2). Impurities: <1 mol % nicotinamide; 0.7 mol % TEA salt: 1.19 (t, 9H), 3.11 (q, 6H). Solvents: 6.3 mol % methanol: 3.25 (s, 3H).

¹³C-NMR (100 MHz, D₂O): 40.0 (CH₂, H-malate), 60.2 (C′), 68.5 (CHOH, H-malate), 69.7 (C3′), 77.4 (C2′), 87.7 (C4′), 99.9 (C1′), 128.4 (C5), 133.9 (C3), 140.4 (C2), 142.6 (C6), 145.6 (C4), 165.7 (CONH2), 176.3 (COO, H-malate), 179.0 (COO, H-malate). Solvents: 48.9 (methanol).

XRD: crystalline (see FIG. 2)

### Example 2b: Nicotinamide-β-D-Ribofuranoside D-Hydrogen Malate, Example 2c: Nicotinamide-β-D-Ribofuranoside DL-Hydrogen Malate, and Example 2d: Nicotinamide-β-D-Ribofuranoside D-Hydrogen Tartrate

The following crystalline nicotinamide-β-D-ribofuranoside salts of the following table were prepared analogously to the method described above:

| | Yield | Mp | Residual Bromide |
|---|---|---|---|
| anion | [%] | [° C.] | (IC) [%] |
| D-hydrogen malate | 60 | 117-117.5 | 0.9 |
| (FIG. 1) | | | |
| DL-hydrogen malate | 67 | 108-109 | 2.3 |
| (FIG. 3) | | | |
| D-hydrogen | 70 | 124-126 | 0.3 |
| tartrate (FIG. 9) | | | Water content: 0.3% |
| | | | determined according to K. |
| | | | Fischer |

### Example 3: Preparation of Nicotinamide-β-D-Ribofuranoside D-Hydrogen Tartrate Monohydrate by Recrystallization of Nicotinamide-β-D-Ribofuranoside D-Hydrogen Tartrate from Water

2.0 g nicotinamide-β-D-ribofuranoside D-hydrogen tartrate having the XRD of FIG. 9 (termed herein as anhydrous) were dissolved in 9 ml water. 70 ml methanol were added to the colorless solution with stirring. After approx. one minute white crystals precipitated. One hour later the formed suspension was filtered. The residue was washed with methanol and dried in vacuo at 35° C. 1.54 g (77%) of a white crystalline powder of the monohydrate was obtained. Water content: 4.24% (determined according to K. Fischer); Mp.: 115-116° C.; IC: Residual bromide: <0.01%. XRD: crystalline (see FIG. 4)

### Example 4: Preparation of Nicotinamide-β-D-Ribofuranoside-2,3,5-Triacetate L-Hydrogen Tartrate and Nicotinamide-β-D-Ribofuranoside-2,3,5-Triacetate D-Hydrogen Tartrate Via Salt Metathesis from Nicotinamide-β-D-Riboside-2,3,5-Triacetate Bromide

### Example 4a: Nicotinamide-β-D-Ribofuranoside-2,3,5-Triacetate L-Hydrogen Tartrate

3.90 g L-tartaric acid were dissolved in 10 ml methanol upon stirring. The solution was cooled down to 0-5° C. 3.64 ml triethylamine were added. The pH value was 4.1. 15 ml of a 1.73 molar solution of triethylammonium L-hydrogen tartrate was obtained.

8.0 g of nicotinamide-2,3,5-tri-O-acetyl-β-D-riboside bromide were suspended in 10 ml methanol upon stirring. 10 ml of the above generated triethylammonium L-hydrogen tartrate solution were added. A white crystalline powder slowly started precipitating. The residue obtained after filtration was dried in vacuo at 35° C. 6.00 g (65.2%) of a white crystalline powder was obtained. Mp. 128° C.; IC: residual bromide <0.1%.

¹H-NMR (400 MHz, D₂O): 2.08, 2.12, 2.15 (3x s, 3x 3H, COCH₃), 4.43 (s, 2H, 2x CHOH, H-tartrate), 4.52 (m, 2H, H5′), 4.88 (m, 1H, H4′), 5.44 (t, 1H, H3′), 5.55 (dd, 1H, H2′), 6.58 (d, 1H, H1′), 8.27 (t, 1H, H5), 8.99 (d, 1H, H4), 9.20 (d, 1H, H6), 9.43 (s, 1H, H2). Impurities: <0.1 mol % nicotinamide; 0.6 mol % TEA salt: 1.21 (t, 9H), 3.13 (q, 6H). Solvents: 2 mol % methanol: 3.27 (s, 3H).

¹³C-NMR (100 MHz, D₂O): 19.8, 19.9, 20.2 (3x COCH₃), 62.6 (C5′), 69.4 (C3′), 72.8 (2x CHOH, H-tartrate), 76.3 (C2′), 82.6 (C4′), 97.3 (C1′), 128.6 (C5), 134.2 (C3), 140.4 (C2), 143.1 (C6), 146.2 (C4), 165.5 (CONH2), 172.3, 172.4, 173.3 (3x CO), 176.3 (2x COO, H-tartrate).

XRD: crystalline (see FIG. 7).

### Example 4b: Nicotinamide-β-D-Ribofuranoside-2,3,5-Triacetate D-Hydrogen Tartrate

The product was prepared analogously to Example 4a using D-tartaric acid. XRD is shown in FIG. 8.

### Example 5: Deacylation of Nicotinamide-β-D-Ribofuranoside-2,3,5-Triacetate L-Hydrogen Tartrate using Sulfuric Acid and Neutralization using Triethylamine

Preparation of a diluted sulfuric acid in methanol: 27 g methanol were cooled down to 0° C. 3.00 g sulfuric acid were added while stirring resulting in a 10% methanolic sulfuric acid.

Deacylation of nicotinamide-β-D-riboside-2,3,5-triacetate L-hydrogen tartrate: 3.00 g nicotinamide-β-D-riboside-2,3,5-triacetate L-hydrogen tartrate were suspended in 15 ml methanol while stirring. After addition of 11.7 g of the above methanolic sulfuric acid a yellowish solution was generated. After stirring at room temperature for 5 days, only product and nicotinamide as impurity were present as detected by thin-layer chromatography.

Conversion to nicotinamide-β-D-riboside L-hydrogen tartrate after neutralization with triethylamine: 1.1 ml triethylamine were added to the above solution in order to adjust pH to about 3.5. 0.85 g L-tartaric acid were added. After addition of 0.8 ml triethylamine, the product started crystallizing. The suspension was stirred for another hour and was then stored for 12 hours in a refrigerator. The formed crystals were filtered off, washed with isopropanol and were dried in vacuo at 30° C. 1.01 g (44.2%) of a white crystalline powder having a melting point of 126-127° C. were obtained.

¹H-NMR (400 MHz, D₂O): 3.82 (dd, 1H, H5′), 3.96 (dd, 1H, H5′), 4.27 (t, 1H, H3′), 4.37-4.45 (m, 2H, H4′, H2′), 4.42 (s, 2H, 2x CHOH, H-tartrate), 6.17 (d, 1H, H1′), 8.20 (t, 1H, H5), 8.90 (d, 1H, H4), 9.19 (d, 1H, H6), 9.52 (s, 1H, H2). Impurities: 3 mol % nicotinamide: 7.85 (m, 1H), 8.56 (m, 1H), 8.77 (d, 1H), 9.00 (s, 1H); 3.4 mol % TEA salt: 1.18 (t, 9H), 3.11 (q, 6H). Solvents: 11.3 mol % methanol: 3.25 (s, 3H).

¹³C-NMR (100 MHz, D₂O): 60.2 (C5′), 69.7 (C3′), 72.8 (2x CHOH, H-tartrate), 77.4 (C2′), 87.6 (C4′), 99.9 (C1′), 128.4 (C5), 133.9 (C3), 140.4 (C2), 142.6 (C6), 145.6 (C4), 165.8 (CONH2), 176.3 (2x COO, H-tartrate). Impurities: 8.2, 46.6 (TEA salt). Solvents: 48.9 (methanol).

### Preparation of Bromide, Chloride And p-Tosylate Salts of Nicotinamide Riboside from Crystalline Salts of Nicotinamide Riboside Hydrogen Malate and Hydrogen Tartrate

### Example 6: Preparation of Nicotinamide-β-D-Riboside Bromide

### Example 6a: Preparation of Nicotinamide-β-D-Riboside Bromide Using Nicotinamide-β-D-Riboside L-Hydrogen Malate as Starting Material and Hydrogen Bromide in Glacial Acetic Acid for Ion Exchange Via Salt Metathesis

5 g (12.88 mmole) nicotinamide-β-D-ribofuranoside L-hydrogen malate were suspended in 20 ml methanol at room temperature. 4.74 g (19.3 mmole, 1.5 equiv.) of a solution of hydrogen bromide in glacial acetic acid (33.3%) were dropped to the white suspension within one hour. During the addition of the acid, a clear solution resulted from which the product started precipitating after the addition of the acid was terminated. The resulting suspension was stirred at room temperature for one hour and subsequently for another hour while cooling with ice water. The obtained white solid in the form of crystals was filtered off and subsequently washed with 14 ml isopropanol and 14 ml acetone. 3.68 g (85.3%) of crystalline nicotinamide-β-D-ribofuranoside bromide having a melting point of 117° C. were obtained. Melting point and IR data were identical with the respective data of a reference example synthesized by known methods.

¹H-NMR (400 MHz, D₂O): 3.87 (dd, 1H, H5′), 4.01 (dd, 1H, H5′), 4.34 (m, 1H, H3′), 4.44 (q, 1H, H4′), 4.52 (t, 1H, H2′), 6.23 (d, 1H, H1′), 8.27 (t, 1H, H5), 8.96 (dt, 1H, H4), 9.24 (d, 1 H, H6), 9.56 (s, 1H, H2). Impurities: <1 mol % nicotinamide; <0.5 mol % malic acid. Solvents: 2.3 mol % methanol.

¹³C-NMR (100 MHz, D₂O): 60.3 (C5′), 69.8 (C3′), 77.4 (C2′), 87.7 (C4′), 100.0 (C1′), 128.5 (C5), 134.0 (C3), 140.4 (C2), 142.7 (C6), 145.7 (C4), 165.8 (CONH₂).

### Example 6b: Preparation of Nicotinamide-β-D-Riboside Bromide Using Nicotinamide-β-D-Riboside L-Hydrogen Malate as Starting Material and Aqueous Hydrobromic Acid for Ion Exchange Via Salt Metathesis

5 g (12.88 mmole) nicotinamide-β-D-ribofuranoside L-hydrogen malate were suspended in 20 ml methanol at room temperature. 3.25 g (19.3 mmole, 1.5 equiv.) of aqueous hydrobromic acid (48% by strength) were dropped to the white suspension within one 20 minutes. During the addition of the acid, a clear solution resulted from which the product started precipitating after the addition of the acid was terminated. The resulting suspension was stirred at room temperature for 10 minutes and subsequently for another two hours while cooling with ice water. The obtained white solid in the form of crystals was filtered off and subsequently washed with 14 ml isopropanol and 14 ml acetone. 3.58 g (83%) of crystalline nicotinamide-β-D-ribofuranoside bromide having a melting point of 117 ° C. in the form of white crystals were obtained. Melting point and NMR data were identical with the respective data of Example 6a.

### Example 7: Preparation of Nicotinamide-β-D-Riboside Chloride Using Nicotinamide-β-D-Riboside L-Hydrogen Malate as Starting Material and Hydrogen Chloride for Ion Exchange Via Salt Metathesis

5 g (12.88 mmole) nicotinamide-β-D-ribofuranoside L-hydrogen malate were suspended in 20 ml methanol. 3.20 ml (19.4 mmole, 1.5 equiv.) of a solution of hydrogen chloride (6.7 mole/kg) in ethanol were added within one hour. During the addition of the acid, a clear solution resulted from which the product started precipitating after the addition of the acid was terminated. The resulting suspension was stirred at room temperature for one hour and subsequently for another hour while cooling with ice water. The obtained white solid in the form of crystals was filtered off and subsequently washed with 14 ml isopropanol and 14 ml acetone. 2.88 g (76.9%) of crystalline nicotinamide-β-D-ribofuranoside chloride having a melting point of 113° C. were obtained. XRD was identical with the XRD of a reference example synthesized by known methods.

### Example 8: Preparation of Nicotinamide-β-D-Riboside Tosylate Using Nicotinamide-β-D-Riboside L-Hydrogen Malate as Starting Material and p-Toluenesulfonic Acid for Ion Exchange Via Salt Metathesis

50 g (128.8 mmole) nicotinamide-β-D-ribofuranoside L-hydrogen malate were suspended in 250 ml methanol. 36.8 g (193 mmole, 1.5 equiv.) of p-toluenesulfonic acid (as monohydrate) were added. A clear solution resulted. The solvent was partially evaporated in vacuo at 40° C., wherein the product started precipitating. 500 ml ethanol were added to the residue. The resulting suspension was stirred at room temperature for one hour. The obtained white solid in the form of crystals was filtered off and subsequently washed with 140 ml isopropanol and 140 ml acetone. 49.8 g (90.7%) of crystalline nicotinamide-β-D-ribofuranoside tosylate having a melting point of 124° C. were obtained.

¹H-NMR (400 MHz, D₂O): 3.81 (dd, 1H, H5′), 3.96 (dd, 1H, H5′), 4.27 (m, 1H, H3′), 4.40 (m, 2H, H4′, H2′), 6.13 (d, 1H, H1′), 8.12 (t, 1H, H5), 8.80 (dt, 1H, H4), 9.13 (d, 1H, H6), 9.46 (s, 1H, H2); tosylate: 2.26 (s, 3H, CH3), 7.21 (d, 2H), 7.52 (d, 2H). Impurities: <1 mol % nicotinamide; malic acid not visible! Solvents: 1 mol % methanol, 0.3 mol % ethanol.

¹³C-NMR (100 MHz, D₂O): 60.2 (C5′), 69.8 (C3′), 77.4 (C2′), 87.7 (C4′), 99.9 (C1′), 128.3 (C5), 133.7 (C3), 139.5 (C2), 142.3 (C6), 145.5 (C4), 165.5 (CONH2); tosylate: 20.5 (CH3), 125.3 (2C), 129.4 (2C), 140.2, 142.5.

XRD: crystalline (FIG. 10)

DSC: Peak from 129-132° C.

### Solubility in Methanol

While 1 g NR·Br needs 50 ml of methanol for dissolving and 1 g NR·Cl needs 40 ml of methanol for dissolving, 1 g NR·p-tosylate needs 15 ml methanol only. The better solubility of NR·p-tosylate compared to NR·Cl may be advantageous for applications where solubility is necessary.

### Example 9: Preparation of Nicotinamide-β-D-Riboside Bromide

### Example 9a: Preparation of Nicotinamide-β-D-Riboside Bromide Using Nicotinamide-β-D-Riboside L-Hydrogen Tartrate as Starting Material and Hydrogen Bromide in Glacial Acetic Acid for Ion Exchange

5 g (12.37 mmole) nicotinamide-β-D-ribofuranoside L-hydrogen tartrate were suspended in 25 ml methanol at room temperature. 4.55 g (18.6 mmole, 1.5 equiv.) of solution of hydrogen bromide in glacial acetic acid (33.3% by weight) were dropped to the white suspension within one hour. The suspension was stirred at room temperature for three hours and subsequently for another hour while cooling with ice water. The obtained white solid in the form of crystals was filtered off and subsequently washed with 14 ml isopropanol and 14 ml acetone. 3.20 g (77.2%) of crystalline nicotinamide-β-D-ribofuranoside bromide having a melting point of 118° C. were obtained. This product was identical with the product from Example 6.

### Example 9b: Preparation of Nicotinamide-β-D-Riboside Bromide Using Nicotinamide-β-D-Riboside L-Hydrogen Tartrate as Starting Material and Aqueous Hydrobromic Acid for Ion Exchange Via Salt Metathesis

The reaction was carried out analogously to Example 6b. Yield 79.4%. Melting point 117-118° C. This product was identical with the product from Example 9a.

### Example 10: Preparation of Nicotinamide-β-D-Riboside Chloride Using Nicotinamide-β-D-Riboside L-Hydrogen Tartrate as Starting Material and Hydrochloric Acid for Ion Exchange Via Salt Metathesis

5 g (12.37 mmole) nicotinamide-β-D-ribofuranoside L-hydrogen tartrate were suspended in 20 ml methanol. 1.75 ml (18.5 mmole, 1.5 equiv.) of hydrochloric acid (32.5%) were added within one hour. During the addition of the acid, a clear solution resulted from which the product started precipitating after the addition of the acid was terminated. The resulting suspension was stirred at room temperature for one hour and subsequently for another hour while cooling with ice water. The obtained white solid in the form of crystals was filtered off and subsequently washed with 14 ml isopropanol and 14 ml acetone. 1.91 g (53.1%) of crystalline nicotinamide-β-D-ribofuranoside chloride having a melting point of 114° C. were obtained. This product was identical to the product from Example 7.

### Example 11: Preparation of Nicotinamide-β-D-Riboside Tosylate Using Nicotinamide-β-D-Riboside L-Hydrogen Tartrate as Starting Material

5 g (12.37 mmole) nicotinamide-β-D-ribofuranoside L-hydrogen tartrate were suspended in 20 ml methanol. 3.54 g (18.6 mmole, 1.5 equiv.) of p-toluenesulfonic acid (as monohydrate) were added. A clear solution resulted. The solvent was evaporated and 5 ml methanol were added to the oily residue. After addition of 20 ml isopropanol, the product started precipitating. The resulting suspension was stirred at room temperature for one hour. The obtained white solid in the form of crystals was filtered off and subsequently washed with 15 ml isopropanol and 15 ml acetone. 4.33 g (82.2%) of crystalline nicotinamide-β-D-ribofuranoside tosylate having a melting point of 120° C. were obtained. XRD was identical with the XRD of the product from Example 8.

### Example 12: Preparation of Nicotinamide Riboside Tosylate

### Example 12a: Preparation of Nicotinamide Riboside Tosylate From a Triflate Salt of Triacetyl Nicotinamide Riboside in Methanol

4 g (7.54 mmole) nicotinamide-β-D-riboside-2,3-5-triacetate triflate were dissolved in 6 ml methanol at room temperature. 1.74 g (9.05 mmol; 1.2 equiv.) p-toluenesulfonic acid (as monohydrate) were added. The yellow solution was stirred overnight at room temperature, wherein educt started precipitating. The solid was filtered off and washed twice with isopropanol. Yield 1.22 g (38%), melting point: 126° C.

¹H-NMR (400 MHz, D₂O): 3.80 (dd, 1H, H5′), 3.94 (dd, 1H, H5′), 4.26 (m, 1H, H3′), 4.38 (m, 2H, H4′, H2′), 6.12 (d, 1H, H1′), 8.08 (t, 1H, H5), 8.78 (dt, 1H, H4), 9.11 (d, 1H, H6), 9.42 (s, 1H, H2); tosylate: 2.23 (s, 3H, CH3), 7.17 (d, 2H), 7.49 (d, 2H). Impurities: <1 mol % nicotinamide. Solvents: 0.5 mol % methanol, 0.1 mol % iso-propanol.

¹³C-NMR (100 MHz, D₂O): 60.2 (C5′), 69.8 (C3′), 77.5 (C2′), 87.7 (C4′), 99.9 (C1′), 128.3 (C5), 133.8 (C3), 139.5 (C2), 142.2 (C6), 145.4 (C4), 165.4 (CONH2); tosylate: 20.5 (CH3), 125.3 (2C), 129.4 (2C), 140.1, 142.5.

### Example 12b: Preparation of Nicotinamide Riboside Tosylate from a Triflate Salt of Triacetyl Nicotinamide Riboside in Ethanol

4 g (7.54 mmole) nicotinamide-β-D-riboside-2,3-5-triacetate triflate were dissolved in 12 ml ethanol at room temperature. 2.90 g (15.1 mmole; 2 equiv.) p-toluenesulfonic acid (as monohydrate) were added. The yellow solution was stirred overnight at room temperature, wherein educt started precipitating. The solid was filtered off and washed twice with isopropanol. Yield 2.03 g (63%), melting point: 102° C.

¹H-NMR (400 MHz, D₂O): 3.78 (dd, 1H, H5′), 3.93 (dd, 1H, H5′), 4.24 (m, 1H, H3′), 4.36 (m, 2H, H4′, H2′), 6.10 (d, 1H, H1′), 8.06 (t, 1H, H5), 8.73 (dt, 1H, H4), 9.08 (d, 1H, H6), 9.40 (s, 1H, H2); tosylate: 2.21 (s, 3H, CH3), 7.14 (d, 2H), 7.47 (d, 2H). Impurities: 4 mol % nicotinamide and impurities in the sugar region. Solvents: 2.7 mol % ethanol, 8.5 mol % iso-propanol, 4.4 mol % acetone.

¹³C-NMR (100 MHz, D₂O): 60.2 (C5′), 69.8 (C3′), 77.5 (C2′), 87.7 (C4′), 99.9 (C1′), 128.3 (C5), 133.6 (C3), 139.5 (C2), 142.2 (C6), 145.4 (C4), 165.3 (CONH2); tosylate: 20.5 (CH3), 125.3 (2C), 129.4 (2C), 140.1, 142.4.

### Example 13a: Preparation of a Co-Crystallized Nicotinamide-β-D-Ribofuranoside (Chloride, Iodide), Wherein Chloride and Iodide are Present in a Ratio of 1.5:1

5 g (12.88 mmole) nicotinamide-β-D-ribofuranoside L-hydrogen malate were dissolved in 1.9 ml (14.4 mmole) Hl (57% in water) (1.1 equivalents) and 7.8 ml (7.2 mmole) 0.92 N HCl in ethanol (0.56 equivalents) at room temperature in a 250 ml round bottom flask. By addition of 10 ml methanol and 26 ml ethanol a bright yellow emulsion was generated, which, by addition of 3 ml of methanol, was nearly completely re-dissolved. Very slow crystallization started (the crystallization rate can be accelerated by addition of seed crystals). The suspension was diluted with further 60 ml ethanol and was stored overnight in a refrigerator. The light-yellow suspension was filtrated and the obtained solid was washed thrice with ethanol. The solid was dried in vacuo at 25° C. Yield: 2.32 g of a yellow fine-crystalline powder; melting point 104° C.

¹H-NMR (400 MHz, D₂O): 3.87 (dd, 1H, H5′), 4.01 (dd, 1H, H5′), 4.34 (m, 1H, H3′), 4.43 (q, 1H, H4′), 4.51 (t, 1H, H2′), 6.23 (d, 1H, H1′), 8.27 (t, 1H, H5), 8.95 (dt, 1H, H4), 9.25 (d, 1H, H6), 9.55 (s, 1H, H2). Impurities: <0.5 mol % nicotinamide; <0.2 mol % malic acid. Solvents: 0.7 mol % ethanol.

¹³C-NMR (100 MHz, D₂O): 60.3 (C5′), 69.8 (C3′), 77.4 (C2′), 87.7 (C4′), 100.0 (C1′), 128.5 (C5), 134.0 (C3), 140.4 (C2), 142.7 (C6), 145.7 (C4), 165.8 (CONH2).

The following table shows the results when nicotinamide-β-D-ribofuranoside L-hydrogen malate used as starting material is subjected to a mixture of HCl (32.5% by weight in water, respectively 1 N HCl in ethanol) and Hl (57% by weight in water) according to step (A) of the method as defined in the first aspect of the invention:

| | | | | chloride/iodide |
|---|---|---|---|---|
| Example | 32.5% HCl | 1N HCl | 57% HI | ratio determined |
| 13 | (equivalents) | (equiv.) | (equiv.) | by IC analysis |
| b | 1 | — | 1.1 | 3:1 |
| c | — | 0.92 | 0.5 | ~5:1 |
| d | — | 0.92 | 1.1 | 3:1 |
| e | — | 0.74 | 1.1 | 2:1 |
| a | — | 0.56 | 1.1 | 1.5:1 |

The table shows that the iodide content in the crystals correlates with iodide content in the solution used for counter-ion exchange. However, the general tendency is that less iodide is incorporated within the crystal lattice than is present in the solution relative to the chloride content.

The following table shows the melting points measured at a heating rate of 1° C./min:

| | Melting point |
|---|---|
| Example | (heating rate 1° C./min) |
| a | 104 |
| b | 108.5 |
| c | 110 |
| d | 108.5 |
| e | 104.5 |

The following table shows solubilities in mL solvent per g of the co-crystallized nicotinamide-β-D-ribofuranoside (chloride/iodide) relative to nicotinamide-β-D-ribofuranoside L-hydrogen malate and nicotinamide-β-D-ribofuranoside chloride in methanol:

| NR⁺ salts | Solubility in MeOH |
|---|---|
| L-hydrogen malate | 500 |
| chloride | 40 |
| chloride:iodide = ~5:1 | 23 |
| chloride:iodide = 3:1 | 21 |
| chloride:iodide = 2:1 | 17 |
| chloride:iodide = 1.5:1 | 16 |

The solubility of the co-crystallized salts is significantly better than that of the pure chloride. The solubility increases with increasing iodide content. Accordingly, the co-crystallized nicotinamide-β-D-ribofuranoside (chloride/iodide) should allow tailor-made solubilities depending on the chloride/iodide ratio. This may be advantageous in view of applications.

### Example 14: Preparation of Nicotinamide-β-D-Ribofuranoside Bromide from Nicotinamide-β-D-Ribofuranoside Tosylate

1.5 g (3.52 mmole) nicotinamide-β-D-ribofuranoside tosylate prepared according to Example 12a were suspended in 7.5 ml methanol in a 50 ml round bottom flask. 1.25 ml (7.14 mmole) HBr (33% in glacial acetic acid) were added at room temperature. The suspension started dissolving. Remaining solids were dissolved upon slight warming. Product started precipitating. The suspension was stirred for 1 hour at room temperature. The white suspension was filtrated and the obtained solid was washed with 2 ml methanol, subsequently with 5 ml of a 1:1 mixture of methanol and ethanol and finally with 5 ml of ethanol. The solid was dried in vacuo at 25° C. Yield: 0.77 g (65.3%) of a white crystalline solid; melting point 120.5° C.

¹H-NMR (400 MHz, D₂O): 3.87 (dd, 1H, H5′), 4.01 (dd, 1H, H5′), 4.33 (m, 1H, H3′), 4.44 (q, 1H, H4′), 4.50 (t, 1H, H2′), 6.23 (d, 1H, H1′), 8.26 (t, 1H, H5), 8.95 (dt, 1H, H4), 9.24 (d, 1H, H6), 9.56 (s, 1H, H2). Impurities: <0.1 mol % nicotinamide; <0.1 mol % residual tosylate. Solvents: 0.7 mol % methanol.

¹³C-NMR (100 MHz, D₂O): 60.3 (C5′), 69.8 (C3′), 77.4 (C2′), 87.7 (C4′), 100.0 (C1′), 128.5 (C5), 134.0 (C3), 140.4 (C2), 142.7 (C6), 145.7 (C4), 165.8 (CONH2).

### Example 15: Preparation of Nicotinamide-β-D-Ribofuranoside Chloride from Nicotinamide-β-D-Ribofuranoside Tosylate

1.5 g (3.52 mmole) nicotinamide-β-D-ribofuranoside tosylate prepared according to Example 12a were suspended in 6 ml methanol in a 50 ml round bottom flask. The suspension was heated to 60° C., wherein the solid was completely dissolved. 1.00 ml (10.6 mmole) HCl 32.5% were added. Product started precipitating upon seeding. The suspension was stirred for 3 hours at room temperature. The white suspension was filtrated and the obtained solid was washed thrice with 2 ml ethanol, respectively. The solid was dried in vacuo at 25° C. Yield: 0.57 g (55.8%) of a white crystalline solid; melting point 119° C.

¹H-NMR (400 MHz, D₂O): 3.84 (dd, 1H, H5′), 4.00 (dd, 1H, H5′), 4.30 (m, 1H, H3′), 4.42 (q, 1H, H4′), 4.46 (t, 1H, H2′), 6.20 (d, 1H, H1′), 8.22 (t, 1H, H5), 8.92 (dt, 1H, H4), 9.21 (d, 1H, H6), 9.54 (s, 1H, H2). Impurities: <0.1 mol % nicotinamide; 0.9 mol % residual tosylate. Solvents: 0.6 mol % methanol.

¹³C-NMR (100 MHz, D₂O): 60.3 (C5′), 69.8 (C3′), 77.4 (C2′), 87.7 (C4′), 100.0 (C1′), 128.5 (C5), 134.0 (C3), 140.4 (C2), 142.7 (C6), 145.7 (C4), 165.8 (CONH2).

1. A method of making a nicotinamide-β-D-ribofuranoside salt, comprising step (A):
(A) subjecting nicotinamide-β-D-ribofuranoside hydrogen malate or nicotinamide-β-D-ribofuranoside hydrogen tartrate to salt metathesis comprising counter-ion exchange to afford the nicotinamide-β-D-ribofuranoside salt.

2. A method of making a nicotinamide-β-D-ribofuranoside salt, comprising steps (A) and (B):
(A) subjecting nicotinamide-2,3,5-tri-O-acyl-β-D-ribofuranoside hydrogen malate or nicotinamide-2,3,5-tri-O-acyl-β-D-ribofuranoside hydrogen tartrate to salt metathesis comprising counter-ion exchange to afford a nicotinamide-2,3,5-tri-O-acyl-β-D-ribofuranoside salt;
(B) deacylating the nicotinamide-2,3,5-tri-O-acyl-β-D-ribofuranoside salt to afford the nicotinamide-β-D-ribofuranoside salt;
wherein steps (A) and (B) are carried out simultaneously or step (B) is carried out subsequently to step (A).

3. The method of claim 2, wherein acyl is independently selected from alkyl carbonyl, aryl carbonyl and heteroaryl carbonyl, preferably from C₁₋₁₀ alkyl carbonyl and benzoyl, and is more preferably acetyl, and wherein acyl is optionally independently substituted with one or more substituents selected from: C₁₋₆ alkyl, C₁₋₆ alkoxy, C₁₋₆ thioalkyl, halogen, nitro, cyano, NH(C₁₋₆ alkyl), N(C₁₋₆ alkyl)₂, and SO₂N(C₁₋₆ alkyl)₂.

4. The method of claim 1, wherein the hydrogen malate is D-, L- or DL-hydrogen malate, or wherein the hydrogen tartrate is D-, L- or DL- hydrogen tartrate.

5. The method of claim 1, wherein the nicotinamide-β-D-ribofuranoside hydrogen malate or the nicotinamide-β-D-ribofuranoside hydrogen tartrate, or the nicotinamide-2,3,5-tri-O-acyl-β-D-ribofuranoside hydrogen malate or the nicotinamide-2,3,5-tri-O-acyl-β-D-ribofuranoside hydrogen tartrate is in the form of a crystalline salt.

6. The method of claim 1, wherein the nicotinamide-β-D-ribofuranoside hydrogen malate or the nicotinamide-β-D-ribofuranoside hydrogen tartrate, or the nicotinamide-2,3,5-tri-O-acyl-β-D-ribofuranoside hydrogen malate or the nicotinamide-2,3,5-tri-O-acyl-β-D-ribofuranoside hydrogen tartrate is reacted with an acid.

7. The method of claim 6, wherein pKₐ of the acid is below 2.

8. The method of claim 1, wherein the counter-ion of the salt obtained in step (A) via counter-ion exchange is selected from the group consisting of:
inorganic ions;
carboxylates, optionally substituted with one or more substituents independently selected from the group consisting of carboxyl, hydroxyl, thio, keto, amino, mono C₁₋₆ alkyl, hydroxy C₁₋₆ alkylene and di(C₁₋₆ alkyl) amino;
C₁₋₁₂ alkyl sulfonates; or
arylsulfonates, wherein the aryl moiety is optionally substituted with one or more substituents independently selected from the group consisting of carboxyl, hydroxyl, amino, mono-C₁₋₆ alkyl and di(C₁₋₆ alkyl)amino, halogen, and C₁₋₆ alkyl; and
wherein the counter-ion is not hydrogen tartrate or hydrogen malate.

9. The method of claim 8, wherein the inorganic ion is selected from the group consisting of bromide, chloride, iodide, hydrogen sulfate, sulfate, dihydrogen phosphate, monohydrogen phosphate, phosphate;
the carboxylate is selected from the group consisting of formate, acetate, oxalate, malonate, succinate, fumarate, maleate, citrate, ascorbate, α-ketoglutarate, glucuronate, benzoate and salicylate;
the C₁₋₁₂ alkylsulfonate is selected from the group consisting of mesylate and camsylate; and
the arylsulfonate is selected from the group consisting of besylate and tosylate.

10. The method of claim 1, where the counter-ion is selected from chloride and bromide, preferably chloride.

11. The method of claim 1, wherein the salt metathesis is performed (i) in an alcohol selected from the group consisting of methanol, ethanol, propanol or butanol, or a mixture of two or more thereof, wherein said alcohol or said mixture optionally comprises water, or (ii) in a solvent comprising methanol, ethanol, propanol or butanol, or a mixture of two or more thereof, wherein said solvent or said alcohol optionally comprises water.

12. The method of claim 1, comprising prior to step (A) step (X):
(X) subjecting a salt of nicotinamide-β-D-ribofuranoside and a counter-ion, wherein the counter-ion is selected from the group consisting of Cl⁻, Br⁻, CF₃SO₃⁻, n-C₄F₉SO₃⁻, FSO₃⁻ and ClO₄, to salt metathesis comprising counter-ion exchange using hydrogen malate or hydrogen tartrate as counter-ion to afford the nicotinamide-β-D-ribofuranoside hydrogen malate or nicotinamide-β-D-ribofuranoside hydrogen tartrate to be used in step (A).

13. The method of claim 2, comprising prior to step (A) step (Y):
(Y) subjecting a salt of nicotinamide-2,3,5-tri-O-acyl-β-D-ribofuranoside and a counter-ion, wherein the counter-ion is selected from the group consisting of Cl⁻, Br⁻, CF₃SO₃³¹ , n-C₄F₉SO₃³¹ , FSO₃⁻ and ClO₄, to salt metathesis comprising counter-ion exchange using hydrogen malate or hydrogen tartrate as counter-ion to afford the nicotinamide-2,3,5-tri-O-acyl-β-D-ribofuranoside hydrogen malate or nicotinamide-2,3,5-tri-O-acyl-β-D-ribofuranoside hydrogen tartrate to be used in step (A).

14. A crystalline form of nicotinamide-β-D-ribofuranoside tosylate.

15. A method of making a nicotinamide-β-D-ribofuranoside salt NR⁺Y⁻ from a nicotinamide-β-D-ribofuranoside salt NR⁺X⁻ or from a nicotinamide-2,3,5-tri-O-acyl-β-D-ribofuranoside salt AcONR⁺X⁻, comprising step (A):
(A) subjecting the nicotinamide-β-D-ribofuranoside salt NR⁺X⁻ or the nicotinamide-2,3,5-tri-O-acyl-β-D-ribofuranoside salt AcONR⁺X⁻ to an acid H⁺Y⁻ to afford the nicotinamide-β-D-ribofuranoside salt NR⁺Y⁻ and H⁺X⁻, wherein pKₐ H⁺Y⁻<pKₐ H⁺X⁻.

16. The method of claim 1, wherein in the counter-ion exchange according to step (A) more than one counter-ion is employed.

17. The method of claim 16, wherein two counter-ions are employed.

18. The method of claim 16, wherein the counter-ions are selected from chloride and iodide.

19. A co-crystallized nicotinamide-β-D-ribofuranoside salt, wherein the anions of the salt comprise or consist of chloride and iodide.

20. The co-crystallized nicotinamide-β-D-ribofuranoside salt of claim 19, wherein the molar ratio of chloride to iodide is 5:1, 3:1, 2:1, 1.5:1 or 1:1.

21. The co-crystallized nicotinamide-β-D-ribofuranoside salt of claim 19, wherein the molar ratio of chloride to iodide is 2:1, characterized by a powder X-ray diffraction pattern having peaks substantially as provided in Table 11, ±0.2 degrees two theta, or as provided in FIG. 11.

22. A nutritional supplement or a pharmaceutical composition comprising the co-crystallized nicotinamide-β-D-ribofuranoside salt of claims 19.
